(19) United States
(12) Patent Application Publication
(10) Pub. No.: US 2023/0416703 A1
(43) Pub. Date: Dec. 28, 2023

(54) Methods and Materials for Producing Polyols and Electron Rich Compounds

(71) Applicant: THE TRUSTEES OF PRINCETON UNIVERSITY, Princeton, NJ (US)

(72) Inventors: Yi-Fan Xu, Princeton, NJ (US); Joshua D. Rabinowitz, Princeton, NJ (US); Fabien Letisse, Toulouse (FR); Sarah Johnson, Princeton, NJ (US)

(21) Appl. No.: 18/167,711

(22) Filed: Feb. 10, 2023

Related U.S. Application Data
(63) Continuation of application No. 16/851,970, filed on Apr. 17, 2020, now abandoned.
(60) Provisional application No. 62/836,190, filed on Apr. 19, 2019.

(51) Int. Cl. C12N 9/16; C12N 1/16; C12P 7/18

(52) U.S. Cl. CPC C12N 9/16; C12N 1/16; C12Y 301/0305; C12P 7/18; C12Y 301/03048

(57) ABSTRACT

Methods and materials for producing polyols are provided comprising recombinant microorganisms expressing a Pyp1 polyol phosphatase. Also provided herein are methods and materials for producing electron rich compounds in recombinant microorganisms lacking the DET1 and/or PHO13 genes.

### RELATED APPLICATIONS

This application is a continuation of U.S. application Ser. No. 16/851,970, filed Apr. 17, 2020 which claims the benefit of U.S. Provisional Application No. 62/836,190, filed Apr. 19, 2019, which are herein incorporated by reference in their entirety.

### GOVERNMENT SUPPORT

This invention was made with government support under Grant No. CBET-0941143 and Grant No. MCB-0643859 awarded by the National Science Foundation; Grant No. GM-071508 awarded by the National Institutes of Health; Grant No. DE-SC0002077 awarded by the Department of Energy; and Grant No. FA9550-09-1-0580 awarded by the United States Air Force, Air Force Office of Scientific Research. The government has certain rights in the invention.

### REFERENCE TO THE SEQUENCE LISTING

This application includes a sequence listing submitted electronically, in a file entitled 47799A_SeqListing.XML, created on Feb. 10, 2023 and having a size of 7,238 bytes, which is incorporated by reference herein.

### FIELD OF THE INVENTION

The invention relates to methods and materials for producing polyols and electron rich biofuels.

### BACKGROUND

In Saccharomyces cerevisiae, more commonly known as budding yeast, there are about 900 uncharacterized open reading frames (ORFs). While the enzymatic function is postulated for some of these ORFs, it is typically only partially annotated, making further characterization essential for establishing gene functions. Advances in high-throughput screens have yielded transcriptomic and proteomic data that can prioritize candidate enzymes of unknown function for metabolic analysis. The metabolite profiles of uncharacterized ORFs selected based on sequence homology to known enzymes will inform functional genomics, metabolic engineering, and the development of new yeast strains of biotechnological utility.

There is a long standing interest in bioproduction of various chemicals, including biofuels and biomaterials. Although these processes have been achieved by engineering native or foreign enzymes into target microorganisms, the lack of a holistic understanding of metabolism limits the further optimization of the yield of biofuel and biomaterial production. Most of these biofuels and biomaterials are rich in electrons, rendering the reduction of the substrates important steps in their biosynthesis. Nicotinamide adenine dinucleotide reduced phosphate (NADPH) is the most important reducing power for various biofuels including biodiesel and isobutanol and biomaterials including monomers of polyolesters. To achieve a high yield of these chemicals, NADPH regeneration must be upregulated.

Polyols, the reduced forms of sugars, are an important natural family of carbohydrates (Shindou et al., 1988; Teo et al., 2006). Glycerol, the simplest polyol, is the backbone of phospholipids and is excreted by many microbes in response to stress (Nevoigt and Stahl, 1997; Pahlman et al., 2001). Longer chain polyols include erythritol, ribitol, xylitol, arabitol, sorbitol, and mannitol, all of which exist only in the D-form in nature and are usually found in plants. Due to the inability of most organisms to assimilate long chain polyols into glycolysis, they are often regarded as inert solutes with unclear physiological functions. While the biological function of polyols has remained obscure, they have gained commercial interest in the last decade due to their increased usage as a sugar substitute by the food industry (Bradshaw and Marsh, 1994).

Many fungi species are able to produce polyols via reduction of the corresponding sugar (Chang et al., 2007). To engineer polyol production, the standard approach is to first dephosphorylate the sugar phosphate and then reduce the resulting sugar to the polyol (Moon et al., 2010; Povelainen and Miasnikov, 2006, 2007; Toivari et al., 2010). This engineering approach differs from the natural glycerol excretion pathway, where (L)-glycerol-3-phosphate (a.k.a. sn-glycerol-3-phosphate) is first made from the reduction of dihydroxyacetone phosphate, followed by the dephosphorylation of the phosphorylated polyol (Albertyn et al., 1994; Norbeck et al., 1996). This natural approach avoids making a dephosphorylated sugar that might be consumed by or escape from the cell; however, it has not been feasible as an engineering strategy as a suitable polyol phosphatase has not previously been identified in any species.

Because many enzymes remain unannotated even in the best studied organisms such as Baker's yeast, it is possible that various polyol phosphate phosphatases exist but have yet to be characterized. One approach to metabolic enzyme annotation is to knock out the corresponding gene and assess the metabolome of the resulting strain. In general, substrates of the enzyme are likely to increase and products to decrease. This approach has been used to assign function to a variety of enzymes (Clasquin et al., Current protocols in bioinformatics/editoral board, Andreas D. Baxevanis . . . [et al.] Chapter 14, Unit 14 11, 2012; Clasquin et al., 2011; Fiehn et al., Nat Biotechnol 18, 1157-1161, 2000; Quanbeck et al., Frontiers in plant science 3, 15, 2012; Raamsdonk et al., Nat Biotechnol 19, 45-50, 2001; Saghatelian and Cravatt, ife sciences 77, 1759-1766, 2005; Su et al., J Am Chem Soc 134, 773-776, 2012; Xu et al., Mol Syst Biol 9, 665, 2013; Yonekura-Sakakibara et al., The Plant cell 20, 2160-2176, 2008).

Thus, there exists in the art a need for genetically engineered microorganisms with enhanced capacity for NADPH regeneration and generation of industrially important compounds, such as polyols.

### SUMMARY

In one aspect of the disclosure, there is provided a recombinant S. cerevisiae cell comprising a polynucleotide encoding Pyp1, ortholog thereof, or a variant of Pyp1 at least 70% identical to the amino acid sequence of SEQ ID NO: 1, wherein the polynucleotide is operably linked to a heterologous promoter.

In another aspect of the disclosure, there is provided a recombinant microorganism comprising a polynucleotide encoding Pyp1, ortholog thereof, or a variant of Pyp1 at least 70% identical to the amino acid sequence of SEQ ID NO: 1.

In some embodiments, the microorganism is a fungus or a bacteria. In some embodiments, the fungus is S. cerevisiae.

In some embodiments, the microorganism further comprises a polynucleotide encoding an enzyme with sugar phosphate dehydrogenase activity operably linked to a heterologous promoter.

In another aspect of the disclosure, there is provided a method of producing a polyol comprising: (a) culturing a recombinant microorganism comprising a polynucleotide encoding Pyp1, ortholog thereof, or a variant of Pyp1 at least 70% identical to the amino acid sequence of SEQ ID NO: 1; and optionally (b) separating the polyol from the culture.

In some embodiments, the polynucleotide is operably linked to a heterologous promoter.

In some embodiments, the microorganism is a fungus or a bacteria. In some embodiments, the fungus is S. cerevisiae.

In some embodiments, the polyol is a four, five or six-carbon polyol. In some embodiments, the polyol is selected from the group consisting of erythritol, ribitol, arabitol, mannitol and sorbitol. In specific embodiments, the polyol is sorbitol.

In some embodiments, the recombinant microorganism further comprises a polynucleotide encoding an enzyme with sugar phosphate dehydrogenase activity operably linked to a heterologous promoter.

In another aspect of the disclosure, there is provided a recombinant microorganism comprising a mutation or deletion of the DET1 gene or ortholog thereof and a mutation or deletion of the PHO13 gene or ortholog thereof.

In some embodiments, the recombinant microorganism further comprises an engineered biosynthetic pathway for an electron rich compound.

In some embodiments, the engineered biosynthetic pathway comprises 2 or more enzymes for the production of isobutanol.

In some embodiments, the enzyme with sugar phosphate dehydrogenase activity is a ribitol-5-phosphate dehydrogenase, a xylitol-5-phosphate dehydrogenase, an arabitol-5-phosphate dehydrogenase, and erytritol-4-phosphate dehydrogenase, a sorbitol-6-phosphate dehydrogenase, or a sedoheptitol-7-phosphate dehydrogenase.

In some embodiments, the microorganism is a fungus or a bacteria. In some embodiments, the fungus is Saccharomyces cerevisiae.

In another aspect of the disclosure, there is provided a method of producing an electron rich compound comprising: (a) culturing a Saccharomyces cerevisiae cell comprising a deletion or mutation of the DET1 gene and/or PHO13 gene, and overexpressing one or both of (i) a polynucleotide encoding an enzyme with sugar phosphate dehydrogenase activity and a gene encoding a polyol phosphatase or (ii) and engineered biosynthetic pathway for the electron rich compounds; and optionally (b) separating the electron rich compound from the culture.

In some embodiments, the electron rich compound is selected from the group consisting of polyols, butanol, isobutanol, fatty acids, fatty acid esters, long-chain fatty alcohols, biodiesel and biogas.

In some embodiments, the polyol comprises 4 or more carbon atoms. In some embodiments, the polyol is selected from the group consisting of erythritol, ribitol, arabitol, mannitol and sorbitol.

In some embodiments, the polyol phosphatase is encoded by PYP1 or an ortholog thereof.

In some embodiments, the enzyme with sugar phosphate dehydrogenase activity is derived from a species other than Saccharomyces cerevisiae.

In some embodiments, the Saccharomyces cerevisiae cell is cultured in a medium comprising glucose, cellulose or hemicelluloses as a carbon source.

In certain embodiments, the disclosure provides a method to clean up or eliminate unnecessary and inhibitory polyol phosphates which are generated in metabolic engineering processes where cells possess high sugar phosphate pools under a reducing environment. In various embodiments the method comprises contacting the inhibitory polyolphosphates with a polyol phosphatase, such as Pyp1.

Other features and advantages of the present disclosure will become apparent from the following detailed description. It should be understood, however, that the detailed description and the specific examples, while indicating preferred embodiments of the disclosure, are given by way of illustration only, because various changes and modifications within the spirit and scope of the disclosure will become apparent to those skilled in the art from this detailed description.

### BRIEF DESCRIPTIONS OF THE DRAWINGS

FIG. 1(A) shows metabolite structures associated with metabolic phenotype of pyp1Δ. O8P, ribitol-5P and erythritol-4P accumulated in pyp1 cells grown on glucose. Sorbitol-6P accumulated in pyp1Δ cells in the presence of sorbitol. Glycerol-1-phosphate, but not glycerol-3-phosphate, is an in vitro substrate of the Pyp1 enzyme, indicating that Pyp1 is a D-polyol phosphatase. FIG. 1(B) shows relative quantitation of accumulated metabolites in cells grown on glucose. The y axis represents the ratio of metabolite level in pyp1Δ cells vs. wild type cells (mean±range of N=4 biological replicates). FIG. 1(C) shows the negative ionization mode-extracted ion chromatogram for O8P in pyp1Δ and wild type cells. Inset: Mass spectrum displaying the accurate mass for the parent ion (M) and natural ¹³C abundance ion (M+1) observed for O8P in negative ionization mode via LC/MS. FIG. 1(D) shows a table of [M-H] ions with altered abundance between pyp1Δ and wild type cells.

FIG. 2 demonstrates that Pyp1 is active against compounds with a D-glycerol-3-phosphate tail. Purified Pyp1 phosphatase activity was assayed in the presence of 0.5 mM substrate and 5 mM Mg²⁺ (pH=7.0, 30° C.) by monitoring the appearance of free phosphate or the polyol. The y axis represents specific phosphatase activity (μmol product made per mg of protein per minute) (mean±range of N=2 replicates).

FIGS. 3A-3E show that pyp1Δ cells accumulate sorbitol-6-phosphate, inhibiting phosphoglucose isomerase activity and resulting in growth defect. FIG. 3(A) shows growth of pyp1Δ and wild type cells on sorbitol. Yeast cells grown on YPD medium were switched to minimal media containing 2% sorbitol as the carbon source. The x axis represents days after the switch and the y axis represents optical density (A₆₀₀). FIG. 3(B) shows absolute concentration of sorbitol-6-phosphate in pyp1Δ and wild type cells in the experiment shown in (A). The x axis represents days after the switch and the y axis represents absolute intracellular concentration (mean±range of N=2 replicates). FIG. 3(C) shows the specific growth rate (mean±range of N=2 replicates) for yeast cells expressing A6PR enzyme (yA6PR) and empty vector (control). Yeast cells grown on YPD medium were diluted into minimal media containing galactose as the carbon source. FIG. 3(D) shows metabolome profiling of yA6PR (pink) and control cells (dark blue) grown on galactose indicates an upregulated PPP and downregulated glycolysis. FIG. 3(E) shows metabolome profiling of cells with high (black) and low (grey) Pgi1 induction. In (D) and (E), the y axis represents absolute intracellular concentration, except for the panel displaying the NADPH/NADP⁺ ratio (mean±range of N=2 replicates).

FIG. 4 shows that Sorbitol-6-phosphate and ribitol-5-phosphate are inhibitors of phosphoglucose isomerase. Purified Pgi1's activity was assayed in the presence of 0.6 mM fructose-6-phosphate, 5 mM Mg²⁺ (pH=7.0, 30° C.) and a range of 0.05-5 mM of sorbitol-6P or ribitol-5P by monitoring the appearance of glucose-6-phosphate. The y axis represents relative Pgi1 activity (mean±range of N=2 replicates).

FIGS. 5A-5D show that Pyp1's maintenance on Pgi1's activity is more important at a higher growth rate. FIG. 5(A) shows the ratio of growth rates of cells growing in the presence of ribitol vs. cells growing in the absence of ribitol. Wild type yeast and pyp1Δ cells growing on 1% trehalose in the absence or presence of 1% ribitol (upper panel) were switched to media with trehalose substituted by 1% glucose (lower panel). The y axis represents ratio of growth rates (mean±range of N=2 replicates) of cells growing in the presence of ribitol vs. cells growing in the absence of ribitol. FIG. 5(B) shows the absolute concentration of ribitol-5-phosphate in pyp1Δ and wild type cells in the experiment shown in (A). The x axis represents hours after the switch and the y axis represents absolute intracellular concentration (mean±range of N=2 replicates). FIG. 5(C) shows the relative expression level of PYP1 at different growth rates in C-, N-, P- and S-limited chemostats³³. The x axis represents different growth rates and the y axis represents relative expression level of PYP1. FIG. 5(D) shows the metabolome profiling of pyp1Δ (dark red) and wild type (blue) cells at t=1 h after switching from trehalose+ribitol to glucose+ribitol. The y axis represents absolute intracellular concentration, except for the panel displaying the NADPH/NADP⁺ ratio (mean±range of N=2 replicates).

FIG. 6 shows a schematic of the roles of Pho13 and Det1 in regulation of (NADPH/NAD+) and (NADP+/NADH) levels in Saccharomyces cerevisiae.

FIGS. 7A-7B shows the level of NADPH (FIG. 7A) and NADP+(FIG. 7B) in S. cerevisiae cells lacking the DET1 or PHO13 genes relative to wild-type S. cerevisiae.

FIG. 8 shows a schematic of metabolic pathways leading to the production of polyols and the role of Yn1010w as a polyol phosphatase.

FIGS. 9A-9F illustrate the metabolomic phenotype of pyp1Δ cells. (FIG. 9A). Metabolite profiles from glucose-grown wild type and pyp1Δ yeast cells detected by untargeted negative ion mode LC-high resolution MS. 1, 2, and 3 represent compounds with significantly different signal intensity between wild type and pyp1Δ cells. The x-axis represents the average of signal intensity in WT cells (N=3). The y-axis represents the average of signal intensity in pyp1Δ cells (N=3). Adduct and isotopic peaks were excluded. (FIG. 9B). Impact of ¹³C- and N-labeling on peaks of interest. Compounds 1, 2 and 3 were assigned formula as C₈H₁₇O₁₁P₁, C₅H₁₃O₈P₁ and C₄H₁₁O₇P₁ respectively. (FIG. 9C). Chemical structures of octulose-8P, ribitol- and erythritol-4P. (FIG. 9D). Extracted ion chromatograms of polyol phosphates produced from ¹³C-glucose (above) or by phosphorylation of ¹²C-polyols fed to the pyp1Δ cells (below). pyp1Δ cells growing exponentially on 2% ¹³C-glucose was switched to 2% ¹²C-polyols. Yeast metabolome right before the switch and four hours after the switch were analyzed by LC-MS. Retention time identifies the glucose-derived five carbon polyol phosphate as ribitol-5-phosphate and the four carbon polyol phosphate as erythritol-4P. (FIGS. 9E and F). Extracted ion chromatogram for endogenous ribitol-5P (FIG. 9E) and octulose-8P (FIG. 9 F) compared to synthetic standards.

FIGS. 10A-10B illustrate that Pyp1 hydrolyzes D-polyol phosphates. (FIG. 10A). Purified Pyp1's phosphatase activity was assayed against 0.5 mM ribitol-5P by LC-MS. Incubation with Pyp1 led to the depletion of ribitol-5P (left panel) and the accumulation of ribitol (right panel). (FIG. 10B). Pyp1's D-polyol phosphatase activity was measured in the presence of 0.5 mM substrate and 5 mM Mg²⁺ (pH=7.0, 30° C.) by monitoring the appearance of phosphate or the polyol. The y-axis represents specific phosphatase activity (moles product per min per mg of protein) (mean±range, N=2).

FIG. 11 shows intracellular levels of ribitol-5-phosphate and ribulose-5-phosphate/xylulose-5-phosphate in wild type and zwf1Δ strain. The metabolomes of wild type and zwf1Δ strains growing on 2% glucose were measured by LC-MS. The y-axis represents the relative level for select metabolites (mean±range, N=2).

FIGS. 12A-12D show Pyp1 accelerates yeast growth on or in the presence of polyols. (FIG. 12A). Growth of wild type and pyp1Δ yeast on sorbitol. Yeast cells grown on YPD medium were switched to minimal media containing 2% sorbitol as the carbon source. The x-axis represents days after the switch and the y-axis represents optical density (A₆₀₀). (FIG. 12B). Absolute concentration of sorbitol-6-phosphate in wild type and pyp1Δ cells in the experiment shown in (A). The x-axis represents days after the switch and the y-axis represents absolute intracellular concentration (mean±range, N=2). (FIG. 12C). Growth of wild type and pyp1Δ cells on glycerol and mannitol. Yeast cells grown on YPD medium were switched to medium containing complete supplement mixture (CSM) and 3% glycerol as the carbon source or minimal medium containing 2% mannitol as the carbon source. The x-axis represents time after the switch and the y-axis represents optical density (A₆₀₀). (FIG. 12D). Impact of ribitol in the presence of trehalose as the carbon source. Absolute concentration of ribitol-5P in wild type and pyp1Δ cells (left panel) and their growth rates (right panel) on minimal media containing 1% trehalose+/−1% ribitol.

FIGS. 13A-13C show polyol phosphates inhibit phosphoglucose isomerase in vitro and in growing yeast. (FIG. 13A). Activity of the purified Pgi1 was assayed in the presence of 0.6 mM fructose-6-phosphate (substrate), 5 mM Mg2+ (pH=7.0, 30° C.) and a range of 0.05-5 mM of sorbitol-6P or ribitol-5P by monitoring the appearance of glucose-6-phosphate. The x-axis represents polyol phosphate concentration and the y-axis represents relative Pgi1 activity (mean±range of N=2 replicates). (FIG. 13B). Metabolome profiling of cells with high (100 nM estradiol, comparable to WT protein level, shown in black) and low (1 nM estradiol, ˜1/7 of WT protein level, shown in grey) Pgi1 induction. This experiment is used to define a characteristic low-Pgi metabolome. (FIG. 13C). Metabolome profiling of pyp1Δ (dark red) and wild type (blue) cells at t=1 h after switching from trehalose+ribitol to glucose+ribitol. This experiment shows that PYP1 deletion results, in the presence of ribitol, in a characteristic low-Pgi metabolome.

FIG. 14 shows Pyp1 is highly expressed and functionally important in rapid growth. Relative expression level of PYP1 at different growth rates in C-, N-, P- and S-limited chemostats (Brauer et al., 2008). The x-axis represents different growth rates and the y-axis represents relative expression level of PYP1.

### DETAILED DESCRIPTION

The present disclosure shows that YNL010W, a gene conserved across all fungi species and some plants, encodes a polyol phosphatase (Pyp1) and demonstrates that the enzyme prevents polyol phosphate accumulation in yeast, and that this is physiologically important due to polyols being transition state analogues (Scheme 1) and therefore inhibitors of the essential glycolytic enzyme phosphoglucose isomerase. Thus, through assigning function to this previously unannotated yeast gene, herein both a new glycolytic regulatory mechanism and a promising new enzyme for polyol metabolic engineering are identified.

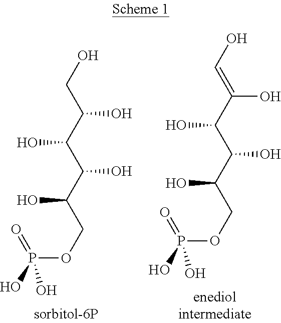

### I. Definitions

The term “sugar phosphate dehydrogenase” refers to an enzyme that catalyzes a chemical reaction that reduces NADP+ to NADPH or NAD+ to NADH and oxidizes a sugar phosphate molecule.

The term “polyol phosphatase” refers to an enzyme that catalyzes a dephosphorylation reaction of a polyol phosphate molecule, producing a polyol and free phosphate.

The term “operably linked” refers to a functional relationship between two or more polynucleotide segments. Typically, it refers to the functional relationship of a transcriptional regulatory sequence to a transcribed sequence. For example, a promoter/enhancer sequence, including any combination of cis-acting transcriptional control elements, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, silencers, insulators, and locus control regions, need not be located in close proximity to the coding sequences whose transcription they enhance

The term “ortholog” refers to genes in different species that evolved from a common ancestral gene by speciation. Typically, orthologs retain the same function in the course of evolution.

The term “electron rich compound” refers to a compound produced by a pathway comprising one or more enzymatic steps requiring a high-energy electron donor such as NADH or NADPH. Electron rich compounds include, but are not limited to, chemical building blocks, polymer starting materials, fine chemicals, and biofuels.

The term “NADPH-derived compound” refers to an electron rich compound produced by a pathway comprising one or more enzymatic steps that utilize NADPH as an electron donor.

The term “biofuel” refers to an electron rich compound, produced by living organism by biomass conversion, that can be burned as a fuel source. Biofuels include, but are not limited to, ethanol, butanol, isobutanol, propanol, isopropanol, methanol, methane, ethane, propane, terpenes, fatty acids, fatty acid esters, biodiesel and biogas. Electron rich biofuels, such as butanol, are important industrial chemicals, useful as a fuel additives, and as feedstock chemicals in the plastics industry, and as a foodgrade extractants in the food and flavor industry. Each year, 10 to 12 billion pounds of butanol, for example, are produced by petrochemical means and the need for this commodity chemical will likely increase.

The term “polyol” refers to an alcohol containing multiple hydroxyl groups. Polyols of the disclosure include, but are not limited to, glycerol, ribitol, xylitol, arabitol, erythritol, and sorbitol.

The term “variant” when used in reference to a polypeptide means a polypeptide that has one or more substitutions, deletions or insertions relative to a parent polypeptide and retains the desired activity. When used in reference to Pyp1, “functional variant” means a variant that retains the ability to catalyze a dephosphorylation reaction of a polyol phosphate molecule, producing a polyol and free phosphate.

As used herein, the term “conservative amino acid substitution” is the replacement of one amino acid with another amino acid having similar properties, e.g. size, charge, hydrophobicity, hydrophilicity, and/or aromaticity, and includes exchanges as indicated below:

| Original | Exemplary |
|---|---|
| Ala (A) | val; leu; ile |
| Arg (R) | lys; gln; asn |
| Asn (N) | gln; his; asp, lys; gln |
| Asp (D) | glu; asn |
| Cys (C) | ser; ala |
| Gln (Q) | asn; glu |
| Glu (E) | asp; gln |
| Gly (G) | ala |
| His (H) | asn; gln; lys; arg |
| Ile (I) | leu; val; met; ala; |
| | phe; norleucine |
| Leu (L) | norleucine; ile; val; |
| | met; ala; phe |
| Lys (K) | arg; gln; asn |
| Met (M) | leu; phe; ile |
| Phe (F) | leu; val; ile; ala; tyr |
| Pro (P) | ala |
| Ser (S) | thr |
| Thr (T) | ser |
| Trp (W) | tyr; phe |
| Tyr (Y) | trp; phe; thr; ser |
| Val (V) | ile; leu; met; phe; |
| | ala; norleucine |

Amino acid residues which share common side-chain properties are often grouped as follows.
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

### II. Pyp1—a Yeast Polyol Phosphatase

Polyols, the reduced forms of sugars, are an important natural family of carbohydrates. Glycerol, the simplest polyol, is the backbone of phospholipids and is excreted by many microbes in response to stress. Longer chain polyols include erythritol, ribitol, xylitol, arabitol, sorbitol, and mannitol, all of which exist only in the D-form in nature and are usually found in plants. Due to the inability of most organisms to assimilate long chain polyols into glycolysis, they are often regarded as inert solutes with unclear physiological functions. While the biological function of polyols has remained obscure, they have gained more commercial interest in the last decade due to their increased usage as a sugar substitute by the food industry because they cannot be readily catabolized by humans and do not contribute to a high blood sugar level.

To achieve a high yield of polyols, the standard approach is to first dephosphorylate a sugar phosphate and then reduce the resulting sugar to the polyol. This engineering approach differs, for example, from the natural glycerol excretion pathway, where sn-(L)-glycerol-3-phosphate is first made from the reduction of dihydroxyacetone phosphate, followed by the dephosphorylation of the phosphorylated polyol. This natural approach is preferable because it avoids making a dephosphorylated intermediate that might be consumed by other pathways or escape the cell; however, it has not been feasible as an engineering strategy because a suitable polyol phosphate phosphatase for most polyols other than glycerol has not previously been identified in any species.

Described herein is the discovery that the previously uncharacterized S. cerevisiae YNL010W gene (also referred to herein as PYP1), which has close orthologs in all fungi species and some plants, encodes a broad-spectrum polyol phosphatase termed Pyp1 herein. The Pyp1 enzyme catalyzes a dephosphorylation reaction with the substrates sorbitol-6-phosphate, mannitol-6-phosphate, ribitol-5-phosphate, xylitol-5-phosphate, arabitol-5-phosphate, erythritol-4-phosphate and glycerol-3-phosphate. It is also demonstrated herein that the Pyp1 enzyme prevents polyol phosphate accumulation in yeast, and that this activity is physiologically important due to polyols being transition state analogues, and therefore inhibitors, of the essential glycolytic enzyme phosphoglucose isomerase, which carries high flux in rapidly growing yeast. The enzyme Pyp1 and its variants and orthologs are of particular utility for their ability to hydrolyze polyol-phosphates containing 4 or more carbon atoms into the corresponding polyol.

A combinatory over-expression of the enzyme encoded by the PYP1 gene and a gene encoding a sugar phosphate dehydrogenase (which converts sugar phosphate to polyol phosphate) represents a novel engineering pathway for polyol production. This approach can be applied to the production of many polyols, by directing the carbon flow in central metabolism toward the production of the corresponding sugar phosphate.

In exemplary embodiments, a recombinant microorganism of the disclosure comprises a polynucleotide encoding Pyp1 or a functional variant of Pyp1 that retains the ability to catalyze a dephosphorylation reaction of a polyol phosphate molecule, producing a polyol and free phosphate. In some or any embodiments, the functional variant comprises an amino acid sequence at least 60%, 65%, 70%, 75%, 80%, 85%, 90% or 95% identical to at least 50 amino acids of residues 1-242 of SEQ ID NO: 1 that retains the ability to catalyze a dephosphorylation reaction of a polyol phosphate molecule. In some embodiments, the Pyp1 variant has been mutated to alter specificity to various substrates. In some embodiments, the Pyp1 protein variant is specific for sorbitol-6-phosphate, mannitol-6-phosphate, ribitol-5-phosphate, xylitol-5-phosphate, arabitol-5-phosphate, erythritol-4-phosphate and/or glycerol-3-phosphate, or combinations thereof.

In some embodiments of the disclosure, a recombinant microorganism comprises a polynucleotide encoding Pyp1 or a variant thereof. The wild type polynucleotide sequence of the S. cerevisiae PYP1 gene is SEQ ID NO: 2. In some embodiments, the PYP1 variant polynucleotide is at least 60%, 65%, 70%, 75%, 80%, 85%, 90%, 95%, 96%, 97%, 98%, or 99% identical to the wild type PYP1 gene (SEQ ID NO: 2). In some embodiments, the PYP1 gene or a variant thereof is operably linked to a heterologous promoter.

In certain embodiments, the polyol phosphatase is utilized to clean up or eliminate unnecessary and inhibitory polyol phosphates which are generated in many metabolic engineering processes where cells possess high sugar phosphate pools under a reducing environment. In certain embodiments, the disclosure provides a method to clean up or eliminate unnecessary and inhibitory polyol phosphates which are generated in many other metabolic engineering processes where cells possess high sugar phosphate pools under a reducing environment. In various embodiments the method comprises contacting the inhibitory polyol phosphates with a polyol phosphatase, such as Pyp1.

### III. Recombinant Microorganisms and Growth Conditions

Microorganisms for polyol or electron rich biofuel production are selected from bacteria, cyanobacteria, filamentous fungi and yeasts. The microorganism used for production is preferably tolerant to the particular product sought to be produced so that the yield is not limited by toxicity.

Suitable microorganisms with a tolerance for the particular product sought to be produced are identified by screening based on the intrinsic tolerance of the strain. The intrinsic tolerance of microbes to a product is measured by determining the concentration of product that is responsible for 50% inhibition of the growth rate (IC₅₀) when grown in a minimal medium. The IC₅₀ values is determined using methods known in the art. For example, the microorganisms of interest are, in various embodiment, grown in the presence of various amounts of product and the growth rate monitored by measuring the optical density at 600 nanometers. The doubling time is calculated from the logarithmic part of the growth curve and used as a measure of the growth rate. The concentration of product that produces 50% inhibition of growth is determined from a graph of the percent inhibition of growth versus the product concentration. In various embodiments, the host strain has an IC₅₀ for product of greater than about 0.5%.

Based on the criteria described above, microbial hosts for the production of electron rich biofuels include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Exemplary host species include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

In some or any embodiments, the recombinant microorganism of the disclosure is a yeast from a genus selected from the group consisting of Saccharomyces, Pichia, Hansenula, Schizosaccharomyces, Kluyveromyces, Yarrowia, and Candida. In some or any embodiments, the fungal species is selected from the group consisting of S. cerevisiae, P. pastoris, H. polymorpha, S. pombe, K. lactis, E lipolytica, and C. albicans. These examples are illustrative rather than limiting.

Fermentation media in the present disclosure contain suitable carbon substrates. Suitable substrates include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate is, in various aspects, a one-carbon substrate such as carbon dioxide, or methanol for which metabolic conversion into key biochemical intermediates has been demonstrated. In addition to one and two carbon substrates, methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth Cl Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Thus, it is contemplated that the source of carbon utilized in the present disclosure encompasses a wide variety of carbon containing substrates and will only be limited by the choice of organism.

In addition to an appropriate carbon source, fermentation media must contain suitable minerals, salts, cofactors, buffers and other components, known to those skilled in the art, suitable for the growth of the cultures and promotion of the enzymatic pathway necessary for electron rich biofuel and polyol production.

### IV. Methods of Producing a Polyol in a Recombinant Microorganism Expressing PYP1

In some embodiments of the disclosure, there is provided a method of producing a polyol in a recombinant microorganism is provided comprising the step of culturing a recombinant microorganism comprising a polynucleotide encoding Pyp1, ortholog thereof, or a variant of Pyp1 at least 70% identical to the amino acid sequence of SEQ ID NO: 1. The method optionally comprises the step of separating the polyol from the culture.

In some embodiments, the polynucleotide encoding Pyp1 is overexpressed in S. cerevisiae. In some embodiments, the polynucleotide encoding Pyp1 is expressed in a host microorganism other than S. cerevisiae (see section II, Recombinant microorganisms). In some embodiments, the polynucleotide encoding Pyp1 is expressed in an organism known to produce high levels of sorbitol-6-phosphate, mannitol-6-phosphate, ribitol-5-phosphate, xylitol-5-phosphate, arabitol-5-phosphate, erythritol-4-phosphate and/or glycerol-3-phosphate, or combinations thereof. In some embodiments, the polynucleotide encoding Pyp1 is overexpressed in Lactobacillus plantarum, which has the capacity to convert fructose-6-phosphate into sorbitol-6-phosphate.

In some embodiments, the polynucleotide encoding Pyp1 is expressed in a microorganism engineered to express the enzymes necessary to produce sorbitol-6-phosphate, mannitol-6-phosphate, ribitol-5-phosphate, xylitol-5-phosphate, arabitol-5-phosphate, and/or erythritol-4-phosphate, or combinations thereof.

### IV. Sugar Phosphate Dehydrogenase Enzymes

In some embodiments, recombinant microorganisms of the disclosure comprise a polynucleotide encoding a sugar phosphate dehydrogenase enzyme to increase production of a particular polyol phosphate. Such embodiments are used in conjunction with a recombinant microorganism comprising a polynucleotide encoding Pyp1, as described above. Thus, increasing production of a particular polyol phosphate provides more substrate for Pyp1 catalysis of the polyol phosphate dephosphorylation reaction. In various embodiments, the sugar phosphate dehydrogenase is a ribitol-5-phosphate dehydrogenase, a xylitol-5-phosphate dehydrogenase, an arabitol-5-phosphate dehydrogenase, an erytritol-4-phosphate dehydrogenase, a glycerol-3-phosphate dehydrogenase, a mannitol-6 phosphate dehydrogenase, or a sorbitol-6-phosphate dehydrogenase. For example, ribitol-5-phosphate dehydrogenase has been found in gram positive pathogens including Haemophilus influenza (accession number Q48230) and Staphylococcus aureus (accession number ADL64305). Mannitol-1-phosphate (same as mannitol-6-phosphate due to the symmetric structure) dehydrogenase (accession number YP_491834.1) and sorbitol-6-phosphate dehydrogenase (accession number YP_490914.1) have been found in various bacteria including Escherichia coli.

In various embodiments, the recombinant microorganism is engineered to overexpress a native gene encoding a sugar phosphate dehydrogenase enzyme or a gene encoding a sugar phosphate dehydrogenase enzyme derived from another species. Methods of obtaining and overexpressing desired genes from a microbial genome are common and well known in the art of molecular biology. For example, if the sequence of the gene is known, suitable genomic libraries are created by restriction endonuclease digestion and are screened with probes complementary to the desired gene sequence. Once the sequence is isolated, the DNA is amplified using standard primer-directed amplification methods such as polymerase chain reaction (U.S. Pat. No. 4,683,202) to obtain amounts of DNA suitable for transformation using appropriate vectors. Tools for codon optimization for expression in a heterologous host are readily available. Some tools for codon optimization are available based on the GC content of the host organism.

### V. Increasing NADPH and NADP+ Levels in Recombinant Microorganisms

The recent development of untargeted liquid chromatography-mass spectrometry (LC-MS) holds the potential in fully mapping microbial metabolism and more completely revealing the key regulatory events in redox balance and energy homeostasis. Using this technique, it was discovered that multiple genes whose knockout directly or indirectly contributes to an upregulated NADPH level, which in turn kinetically and thermodynamically favors production of electron rich bioproducts. Thus, yeast strains lacking these proteins advantageously have an increased cellular pool of NADPH available to generate electron rich compounds. Specifically, the deletion of a protein phosphatase, Pho13, or the deletion of a putative phosphatase, Det1, caused NADPH to accumulate in S. cerevisiae cells. The deletion of Pho13 resulted in an upregulation of NADPH regeneration (NADP+→NADPH) via the acetate production pathway. This upregulation resulted in accumulated NADPH, but not NADP⁺ (i.e., a higher NADPH/NADP+ ratio). The deletion of Det1 resulted in a higher total pool of NADPH and NADP⁺, with a greater effect on NADPH and thus an increased NADPH/NADP+ ratio. Both enzymes have orthologs among fungi species. Deletion of one or two of these enzymes would substantially contribute to a better reducing potential by elevating the pool size of NADPH and the ratio of NADPH/NADP⁺.

A. PHO13

Pho13 in S. cerevisiae is a protein phosphatase with unknown function. It was previously shown that pho13 deletion results in better utilization of xylose (Fujitomi et al., 2012; Ni et al., 2007; Van Vleet et al., 2008), but the mechanism behind the enhanced xylose utilization, however, is unknown. It was also shown that the pho13 deletion strain accumulates acetate in the media (Van Vleet et al., 2008). It was proposed that Pho13 was a protein phosphatase due to its ability to dephosphorylate model phosphopeptides in vitro (Tuleva et al., 1998).

Through the use of untargeted LC-MS measurements, it has been discovered that S. cerevisiae yeast with a pho13 deletion accumulate NADPH compared to a wild-type strain, but not NADP+, NADH or NAD+. Acetate excretion via Ald6 is one of the two major NADPH producing pathways (the other one is oxidative pentose phosphate pathway). Thus, observations described herein that NADPH accumulates are consistent with upregulated acetate excretion. It is also demonstrated herein that oxidative pentose phosphate pathway intermediates, 6-phosphogluconate, glucono-6-lactone-6-phosphate accumulate in the pho13 deletion strain, while pentose-phosphates and sedoheptulose-7-phosphate in the non-oxidative pentose phosphate pathway decrease. These data indicate that the pho13 deletion strain also has an altered pentose phosphate pathway flux. Alteration of both NADPH producing pathways to enhance NADPH production through the deletion or alteration of Pho13 is fundamentally beneficial for metabolic engineering.

In some embodiments, a recombinant microorganism is provided comprising a partially or wholly deleted PHO13 gene or a nonfunctional PHO13 gene (SEQ ID NO: 3).

In some embodiments, partially or wholly deleting or inactivating the PHO13 gene results in an at least a 1.5-fold, 2-fold, 3-fold, 4-fold, 5-fold, or 10-fold increase in cellular NADPH level compared to a wild type strain.

It is understood that reference to the PHO13 gene includes orthologs from species other than S. cerevisiae.

B. DET1

The Det1 (decreased ergosterol transport) protein encoded by the partially annotated ydr051c gene of S. cerevisiae is currently postulated in the literature to be an acid phosphatase involved in sterol transport between the endoplasmic reticulum and plasma membrane. Previous biochemical characterization of DET1 did not identified potential substrates.

The instant disclosure is based in part on discoveries using untargeted LC-MS to reveal that the partially annotated ydr051c gene (also referred to herein as DET1) of S. cerevisiae accumulates NADPH and NADP+ in a prototrophic deletion collection strain where the DET1 open reading frame was knocked out. In a comparable experiment using a yeast strain that overexpresses DET1, the level of NADPH was depleted compared to a wild type strain. Evidence presented herein indicates that the DET1 gene encodes a novel NADP phosphatase, the first of its kind identified in higher eukaryotes. The only previously known NADP phosphatase is from the archaea Methanococcus jannaschii. The enzyme encoded by the DET1 gene may contribute to the regulation of NADP, which is crucial to understanding the pentose phosphate pathway and of industrial importance. The regulatory mechanisms of NAD+/NADP+ metabolic flux have yet to be elucidated.

In some embodiments, a recombinant microorganism is provided comprising a partially or wholly deleted DET1 gene or a mutated (i.e., nonfunctional) DET1 gene. In some embodiments, a recombinant microorganism comprises a partially or wholly deleted DET1 gene or a mutated DET1 gene (SEQ ID NO: 4) and a partially or wholly deleted PHO13 gene or a mutated PHO13 gene.

In some embodiments, partially or wholly deleting or inactivating the DET1 gene results in an at least 1.5-fold, 2-fold, 3-fold, 4-fold, 5-fold, or 10-fold increase in cellular NADPH level compared to a wild type strain.

It is understood that reference to the DET1 gene includes orthologs from species other than S. cerevisiae.

C. Production of Electron Rich Compounds Including Biofuels

As noted above, an increased cellular NADPH level, obtained by inactivation of DET1 and/or PHO13, is advantageously utilized to increase the cellular pool of NADPH available to generate electron rich compounds including biofuels. Recombinant organisms containing the necessary genes that will encode the enzymatic pathway for the conversion of a fermentable carbon substrate to a desired end product such as an electron rich biofuel is constructed using techniques well known in the art. For example, U.S. Pat. Nos. 7,851,188 and 7,993,889 (incorporated by reference) describe methods and materials to produce isobutanol in a microorganism. Pathways to a very large number of other value-added compounds are known in the art.

D. Elimination of Undesired Polyol Phosphastes in Metabolic Engineering

Polyol phosphates, at high concentration, are also known to be inhibitory to primary metabolism because they structurally mimic the enediol intermediate of key enzymes (phosphoglucose isomerase, ribose-phosphate isomerase, triose-phosphate isomerase). Polyol phosphates are highly produced in many metabolic engineering processes where highly concentrated sugar phosphates are reduced to polyol phosphates.

In plants, ribulose-1,5-bisphosphate carboxylase/oxygenase (RuBisCO) has an enediol intermediate and is known to be inhibited by 2-carboxy-D-arabitol-1-phosphate (CA1P) (Andralojc et al., Biochem J. 304 (Pt 3):781-6, 1994). The corresponding 2-carboxy-D-arabitol-1-phosphatase is activated by light and controls cellular level of CA1P. Thus, to further improve RuBisCO activity in plants, maintaining a low intracellular level of CA1P and other polyol phosphates will be helpful. Pyp1 and its homologues can be engineered into plants and provide an alternative way to activate RuBisCO by dephosphorylating the inhibitory polyol phosphates into the inactivated polyol forms.

In metabolic engineering for various bioproducts, the cytosol of hosting cells is usually kept highly reduced to maintain a fast turnover rate from substrate to synthesize reduced products (for example, biodiesel, bioethanol, bioisoprene, fatty acids, other biofuels and biomaterials). Thus, cells are usually engineered to maintain a high NADPH/NADP+ or NADH/NAD+ ratio. High ratios of these cofactors can also occur if redox is not optimally balanced in the engineered cells. Irrespective of its cause, a high redox potential will cause the cells to generate some undesired byproducts, such as polyols (reduced from sugar) and polyol phosphates (reduced from sugar phosphates or phosphorylated from polyols). Polyol phosphates are highly inhibitory because they are transition state analogues of key sugar phosphate isomerases (phosphoglucose isomerase, triose phosphate isomerase and ribose phosphate isomerase).

Recombinant expression of a polyol phosphatase, such as Pyp1 or a homolog or derivative thereof, provides a useful method to avoid the accumulation of polyol phosphates, relieving the inhibition of the key nodes in glycolysis, the pentose phosphate pathway, or carbon fixation pathways. In some embodiments, expression of Pyp1 is useful in enhancing rates or yields of glucose- or xylo se-based fermentation processes, carbon-fixation limited plant or microbial growth or biomass accumulation processes, or other processes requiring high glycolysis, pentose phosphate pathway, or carbon fixation fluxes.

### VI. Separation of Products from Culture Media

The produced electron rich biofuel or polyol are isolated from the medium using methods known in the art. For example, in various embodiments, solids are removed from the fermentation medium by centrifugation, filtration, decantation, or the like. Then, the desired product is isolated from the medium, which optionally has been treated to remove solids as described above, using methods such as distillation, liquid-liquid extraction, or membrane-based separation. Because electron rich biofuels such as isobutanol form a low boiling point, azeotropic mixture with water, distillation can be used to separate the mixture up to its azeotropic composition. Distillation is, in various embodiments, used in combination with another separation method to obtain separation around the azeotrope. In various embodiments, methods that are used in combination with distillation to isolate and purify electron rich biofuels include, but are not limited to, decantation, liquid-liquid extraction, adsorption, and membrane-based techniques. Additionally, electron rich biofuels are, in various embodiments, isolated using azeotropic distillation using an entrainer (see for example Doherty and Malone, Conceptual Design of Distillation Systems, McGraw Hill, New York, 2001).

An isobutanol-water mixture forms a heterogeneous azeotrope so that distillation is used in various embodiments in combination with decantation to isolate and purify the isobutanol. In this method, the isobutanol containing fermentation broth is distilled to near the azeotropic composition. Then, the azeotropic mixture is condensed, and the isobutanol is separated from the fermentation medium by decantation. The decanted aqueous phase is optionally returned to the first distillation column as reflux. The isobutanol-rich decanted organic phase is optionally further purified by distillation in a second distillation column.

Isobutanol is, in various embodiments, isolated from the fermentation medium using liquid-liquid extraction in combination with distillation. In this method, the isobutanol is extracted from the fermentation broth using liquid-liquid extraction with a suitable solvent. The isobutanol-containing organic phase is then distilled to separate the isobutanol from the solvent.

Distillation in combination with adsorption is also used in various embodiments to isolate isobutanol from the fermentation medium. In this method, the fermentation broth containing the isobutanol is distilled to near the azeotropic composition and then the remaining water is removed by use of an adsorbent, such as molecular sieves (Aden et al. Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation is also used in various embodiments to isolate and purify the isobutanol from the fermentation medium. In this method, the fermentation broth containing the isobutanol is distilled to near the azeotropic composition, and then the remaining water is removed by pervaporation through a hydrophilic membrane (Guo et al., J. Membr. Sci. 245, 199-210 (2004)).

### EXAMPLES

The following examples are provided for illustration and are not in any way to limit the scope of the invention.

### Example 1

### A Yeast Pyp1 Mutant Accumulates Octulose-8-Phosphate and Polyol Phosphates

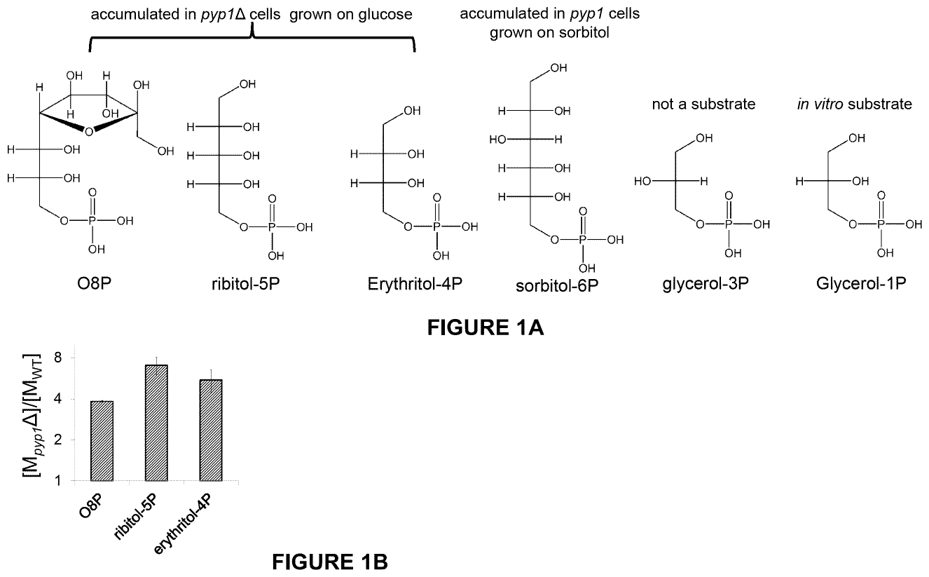

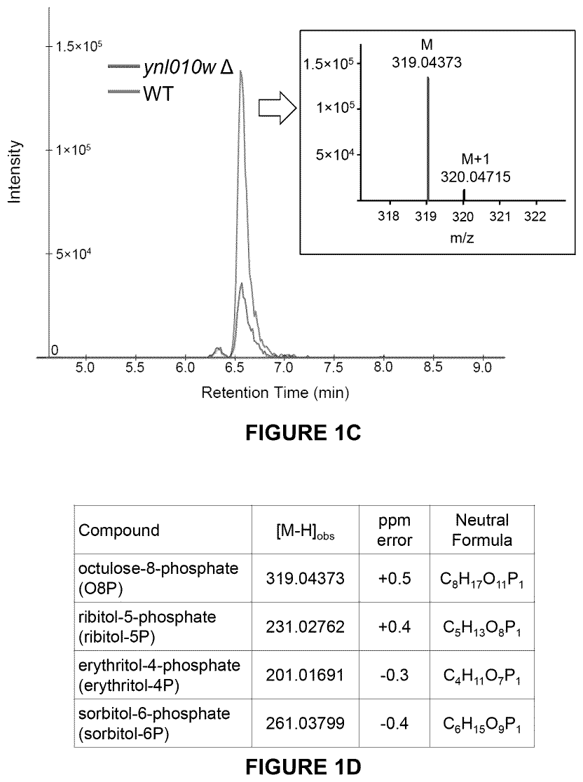

The study described herein was initiated via a metabolomics screen of yeast deletion strains with genes of unknown function. Yeast metabolome was measured by liquid chromatography-mass spectrometry (LC-MS). It was found that the deletion of PYP1, formerly known as the uncharacterized gene YNL010W, led to the accumulation of three metabolites in yeast grown on glucose (FIG. 1).

### Materials and Methods

### Yeast Strains and Media

Yeast strains were derived from prototrophic S288C. Prototrophic deletions were created by homologous recombination using the allele amplified by PCR from the synthetic genetic analysis (SGA) deletion set (Tong et al., 2001). A Pgi1-inducible strain was generated as described by McIsaac et al., Nucleic Acids Res 2013, 41, e57. Briefly, human hormone estradiol was used to induce PGI1 specifically via insertion of a synthetic promoter involving chimeric transcription factor-estrogen receptor in front of PGI1. 1 nM and 5 nM estradiol were added as low and high expression levels of yeast Pgi1, resulting in a 2.5-fold difference in Pgi1 protein level.

Cells were grown in minimal media comprising 6.7 g/L Difco Yeast Nitrogen Base without amino acids plus 2% (w/v) glucose or sorbitol. Glycerol medium was composed of 6.7 g/L Difco Yeast Nitrogen Base without amino acids, 0.79 g/L Complete Supplement Mixture (Sunrise Science, San Diego, CA) and 3% (v/v) glycerol. Trehalose minimal media were prepared by mixing 6.7 g/L Difco Yeast Nitrogen Base without amino acids and 1% (w/v) trehalose, and adjusting pH to 4.8 by adding succinic acid.

### Yeast Culture Conditions and Extraction

The metabolome of batch culture Saccharomyces cerevisiae was characterized as described by Xu et al., Molecular cell 2012, 48, 52. Briefly, saturated overnight cultures were diluted 1:30 and grown in liquid media in a shaking flask to A600 of ˜0.6. A portion of the cells (3 mL) was filtered onto a 50 mm nylon membrane filter (Millipore, Billerica, MA), which was immediately transferred into −20° C. extraction solvent (40:40:20 acetonitrile/methanol/water). For carbon upshift, 100 mL of cell culture grown on trehalose at A600 of ˜0.6 was poured onto a 100 mm cellulose acetate membrane filter (Sterlitech, Kent, WA) resting on a vacuum filter holder with a 1000 mL funnel (Kimble Chase, Vineland, NJ) and was washed with 100 mL pre-warmed (30° C.) glucose minimal medium. Immediately after the wash media went through, the filter was taken off the holder and the cells were washed into a new flask containing 100 mL pre-warmed (30° C.) glucose minimal medium. Samples were then taken at the indicated time points after the switch and filtered and quenched as described above.

### LC/MS Metabolite Measurement

Cell extracts were analyzed by reversed phase ion-pairing liquid chromatography (LC) coupled by electrospray ionization (ESI) (negative mode) to a high-resolution, high-accuracy mass spectrometer (Exactive; Thermo Fisher Scientific, Waltham, Massachusetts) operated in full scan mode at 1 s scan time, 10⁵ resolution at m/z 200. Peaks differing between wild-type and pyp1Δ strain were determined using the in-house developed, open-source software MAVEN (Lu et al., 2010; Melamud et al., 2010). Compounds' identities were verified by mass and retention time matched to authenticated standards. Isomers are reported separately only where they are fully chromatographically resolved. Differences in metabolome between wild type and pyp1Δ strains were tested for significance using Student's T-test. The resulting P-values were then corrected using the Benjamini-Yekutieli False Discovery Rate (FDR) model (Benjamini and Daniel, Ann Stat 20, 1165-1188, 2001)

Absolute intracellular metabolite concentrations in steadily growing S. cerevisiae were determined as described by Bennett et al., Nature Protocols 2008, 3, 1299. Metabolite concentrations after perturbations were computed based on fold-change in ion counts relative to steadily-growing cells (grown and analyzed in parallel) multiplied by the known absolute concentration in the steadily growing cells, as determined using an isotope ratio-based approach.

The number of C and N atoms in each accumulated compound was determined by the method described in Hegeman et al., Anal Chem 2007, 79, 6912. Yeast batch cultures were grown with uniformly labeled glucose or ammonium sulfate (Cambridge Isotopes, Andover, MA) for >20 generations to ensure complete labeling of the metabolome.

### Results

The formulae of the three metabolites identified in the PYP1 deletion strain were obtained by labeling cells with ¹³C and ¹⁵N²⁰ and observing no shift from nitrogen labeling and a shift of +8, +5, or +4 daltons from carbon labeling (Figure S1A). Exact masses of these compounds matched putative formulae of C₈H₁₇O₁₁P₁, C₅H₁₃O₈P₁ and C₄H₁₁O₇P₁. Upon searching these formulae in the KEGG database, matching metabolites were identified as octulose-8-phosphate (O8P) for C₈H₁₇O₁₁P₁, ribitol-5-phosphate (ribitol-5P)/arabitol-5-phosphate (ao15P)/xylitol-5-phosphate (xo15P) for C₅H₁₃O₈P₁, and erythritol-4-phosphate (erythritol-4P) for C₄H₁₁O₇P₁.

The existence of octulose-8-phosphate (O8P) in yeast was reported previously. Indeed, the chromatographic retention time of the accumulated C₈H₁₇O₁₁P₁ exactly matched the synthesized O8P standard. To find the identity of the five- and four-carbon compounds, an isotope labeling experiment was performed that involved switching yeast cells from U—¹³C-glucose to unlabeled ribitol, arabitol, xylitol or erythritol (Figure S1B). Although baker's yeast cannot utilize these polyols as effective carbon sources, they can transport and phosphorylate them slowly in the absence of glucose. Feeding the above-identified five and four-carbon polyols to yeast resulted in the accumulation of intracellular metabolites with masses exactly matching formulae of the above polyol phosphates. Moreover, those polyol phosphates derived from ribitol and erythritol exactly matched the chromatographic retention time of the endogenously accumulated C₅H₁₃O₈P₁ and C₄H₁₁O₇P₁, respectively (Figure S1C). Feeding of arabitol and xylitol resulted in the accumulation of polyol phosphates with different retention times (Figure S1C). Thus, the five- and four-carbon compounds accumulated in the yn1010w deletion strain were identified as ribitol-5P and erythritol-4P.

While ribitol-5P and erythritol-4P are polyol phosphates, O8P is a sugar phosphate. The most common conformer of the seven carbon sugar sedoheptulose and its derivatives in solution is β-furanose. It is likely that O8P has a similar β-furanose ring structure (FIG. 1A), which has three carbons (6′-8′) of octulose out of the ring, forming a D-glycerol-3-phosphate-like tail. Interestingly, this moiety was conserved in all three accumulated metabolites (FIG. 1A), indicating that the enzyme encoded by YNL010W is a non-specific D-polyol phosphatase. Although D-polyols are excretion products of fungi and plants, polyol phosphates were not previously thought to be an intermediate in this pathway (sugar phosphate→sugar→polyol). Thus, this newly identified enzyme is termed polyol phosphatase 1 (Pyp1).

### Example 2

### Pyp1 is a D-polyol Phosphatase

To determine if the accumulated compounds were indeed the substrates of Pyp1, O8P and ribitol-5P were synthesized, and the biochemical activity of recombinant Pyp1 on these compounds was tested.

### Materials and Methods

### Synthesis of ribitol-5-phosphate and octulose-8-phosphate

Synthesis of ribitol-5-phosphate was performed as described by Egan et al., Journal of the American Chemical Society 1982, 104, 2898. Synthesis of D-Glycero-D-Altro-Octulose-8-phosphate was performed enzymatically as described by Kapuscinski et al., Carbohydrate research 1985, 140, 69.

### Results

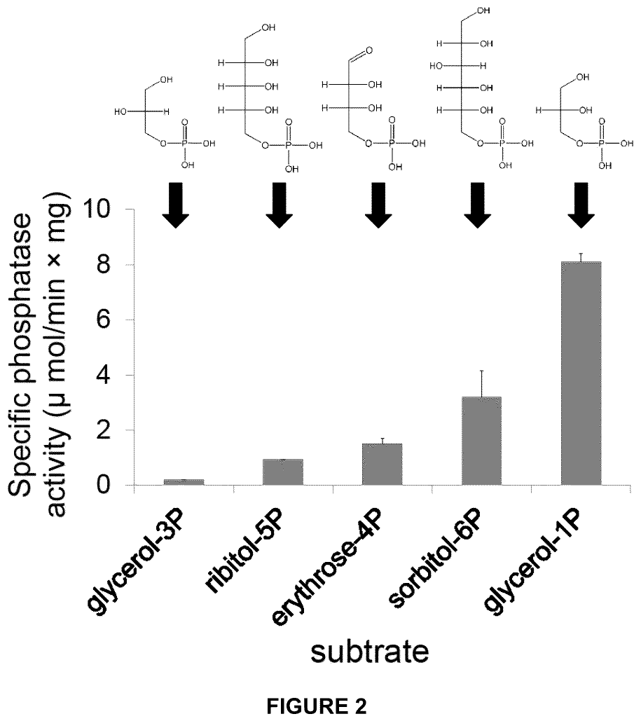

Incubation with Pyp1 led to the depletion of ribitol-5P and the accumulation of ribitol (FIG. 2). No detectable phosphatase activity on O8P was observed, however. O8P is an intermediate of the non-oxidative pentose phosphate pathway (PPP) with a very slow production and consumption rate. Thus, it is likely that Pyp1's activity on O8P is also low, or at least below the detection limit of the assay used. It is also possible that Pyp1 needs to bind an activator or highly active substrate first in order to act on O8P.

To test if Pyp1 is indeed a D-polyol phosphatase with no specificity for the length of the overall carbon chain, its activity against sorbitol-6-phosphate (sorbitol-6P), L-glycerol-3-phosphate (glycerol-3P, the common sn-glycerol-3-phosphate) and D-glycerol-3-phosphate (glycerol-1P, sn-glycerol-1-phosphate) was also measured. Incubation with Pyp1 led to the depletion of sorbitol-6P and glycerol-1P, but not glycerol-3P (FIG. 2). Thus, unlike the known glycerol-3-phosphatases (Hor2 and Rhr2) in the glycerol biosynthetic pathway, which act on both glycerol-3P and glycerol-1P, Pyp1 is specific to D-polyol phosphates.

### Example 3

### A Pyp1 Deletion Mutant Accumulates Sorbitol-6-Phosphate and Fails to Grow on Sorbitol

Although D-polyol phosphates were not thought to be present in baker's yeast, they have been discovered in other fungi species (see Jennings, Adv Microb Physiol 1984, 25, 149). There are two likely routes for the in vivo biosynthesis of these compounds. The first involves the reduction of the corresponding sugar phosphate and the second involves phosphorylation of polyols. Both of these activities were discovered in fungi but the responsible genes have yet to be discovered. As there are no detectable polyols except glycerol in baker's yeast, polyol phosphates in cells grown on glucose as the sole carbon source are more likely made from the reduction of sugar phosphates. In more distant organisms, ribitol-5-phosphate dehydrogenase has been found in gram positive pathogens including Haemophilus influenza and Staphylococcus aureus. Mannitol-1-phosphate (same as mannitol-6-phosphate due to the symmetric structure) dehydrogenase and sorbitol-6-phosphate dehydrogenase have been found in various bacteria, including Escherichia coli. None of these enzymes have homologs in yeast, so the responsible enzyme for polyol phosphate dehydrogenase activity remains unknown. It was demonstrated herein, however, that deletion of Zwf1, the first step in the oxidative PPP, resulted in the disappearance of ribitol-5-phosphate (FIG. 2), presumably due to the strain's inability to synthesize ribulose-5-phosphate. This confirms that ribitol-5-phosphate was made from the reduction of ribulose-5-phosphate in yeast grown on glucose.

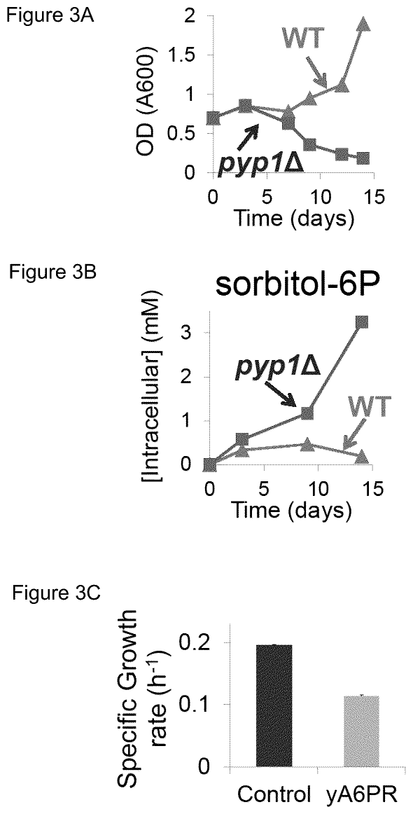

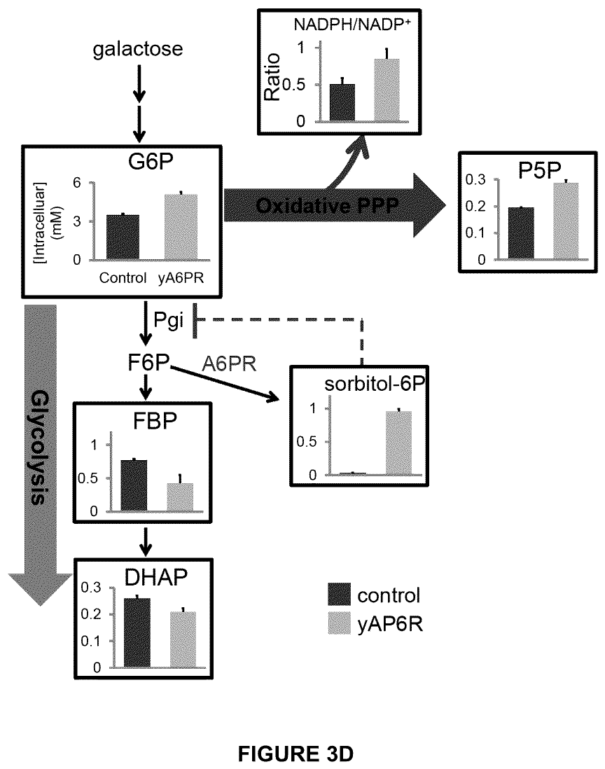

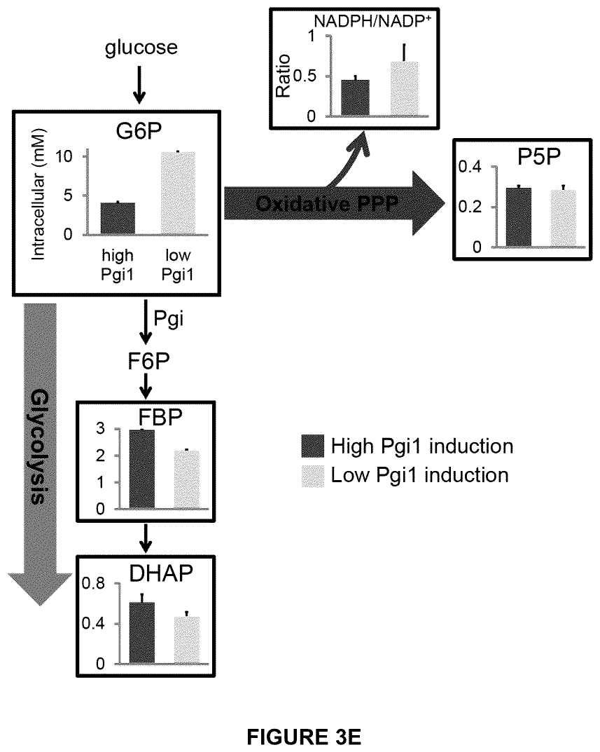

When polyols are present in the growth environment, polyol phosphates can be made from the phosphorylation of polyols. Of all the natural long-chain polyols, sorbitol is the only one known to be able to support yeast growth as the sole carbon source. Unlike bacteria, which use a phosphotransferase system, yeast utilize sorbitol by first oxidizing it into fructose. Because yeast could phosphorylate various polyols in the absence of glucose, it was hypothesized that sorbitol-6P could be made in yeast grown on sorbitol. To test this, both wild type and pyp1Δ strains were grown on minimal media containing sorbitol as the sole carbon source. It was determined that while wild type cells managed to grow after a long lag phase, pyp1Δ cells could not grow (FIG. 3A). Metabolome profiling revealed that sorbitol-6P levels in the pyp1Δ strain were much higher than the wild type strain (FIG. 3B). These results confirmed that Pyp1 dephosphorylates sorbitol-6P in vivo and suggested that sorbitol-6P is toxic to yeast cells.

### Example 4

Sorbitol-6-phosphate Slows Yeast Growth Due to its Inhibition on Phosphoglucose Isomerase

### Materials and Methods

In the initial screening of Pyp1's phosphatase activity, the gene encoding Pyp1 was amplified by PCR using S. cerevisiae genomic DNA. The amplified fragments were cloned into a modified pET15b vector (Novagen, Darmstadt, Germany) and overexpressed in the E. coli BL21(DE3) Gold strain (Stratagene, La Jolla, California) as previously described by Kuznetsova et al., J Biol Chem 2010, 285, 21049. The recombinant proteins were purified using metal ion affinity chromatography on nickel affinity resin (Qiagen, Hilden, Germany) to high homogeneity and stored at −80° C. Purified Pyp1 was screened for the presence of phosphatase activity against the general phosphatase substrate p-nitrophenyl phosphate (pNPP) and 90 phosphorylated metabolites as described previously by Kuznetsova et al., J Biol Chem 2006, 281, 36149.

For the determination of kinetic parameters (K m and h at) of Pyp1, a yeast Open Reading Frame (ORF) strain with an expression vector containing C-terminal His-tagged Pyp1 (Open Biosystems, Thermo Fisher Scientific, San Jose, CA) was grown on galactose to induce Pyp1 expression. The resulting cells were lysed using glass beads and His-tagged Pyp1 was purified using Qiagen Ni-NTA spin columns follow the protocol provided by Qiagen. Phosphatase activity against ribitol-5-phosphate, sorbitol-6-phosphate, and octulose-8-phosphate was determined by monitoring the increase of ribitol, sorbitol or octulose using LC-MS. The reaction mixture contained 100 mM Tris-HCl at the PH of 8.0, 10 mM MgCl₂ and a range of substrate concentrations (0.01 to 5 mM). Kinetic parameters were calculated by non-linear regression analysis of raw data to fit to the Hill equation using the GraphPad Prism Software (GraphPad Software, San Diego, CA).

For the determination of IC₅₀ of sorbitol-6-phosphate and ribitol-5-phosphate on phosphoglucose isomerase, yeast Pgi1 was purchased from Sigma Aldrich (St. Louis, MO). Phosphoglucose isomerase activity was determined by adding fructose-6-phosphate and monitoring the appearance of glucose-6-phosphate using LC/MS. It was determined that the LC/MS-based assay is consistently more sensitive and accurate than the colometric-based assay, which involves coupling the Pgi1 activity with glucose-6-phosphate dehydrogenase activity and monitoring the appearance of NADH. The reaction mixture contained 100 mM Tris-HCl at a pH of 8.0, 10 mM MgCl₂, 0.6 mM fructose-6-phosphate (concentration in cells grown exponentially on glucose), and a range of sorbitol-6-phosphate and ribitol-5-phosphate concentrations (0.05 to 5 mM). The resulting data were again fitted to the Hill equation using the GraphPad Prism Software.

### Results

To investigate the physiological effect of sorbitol-6P, apple aldose-6-phosphate reductase (A6PR), which converts fructose-6-phosphate (F6P) to sorbitol-6P, was expressed under a gal promoter on a high copy plasmid. The resulting cells (yA6PR) and cells transformed with the same plasmid but without the A6PR gene (control cells) were grown on galactose. It was determined that yA6PR cells showed a consistently slower growth rate than the control cells (FIG. 3C) and accumulated ˜1 mM intracellular sorbitol-6P (FIG. 3D). Metabolome profiling revealed that yA6PR cells also accumulated glucose-6-phosphate (G6P) while manifesting lower levels of fructose-1,6-bisphosphate (FBP) and dihydroxyacetone phosphate (DHAP) (FIG. 3D), suggesting that a step between G6P and FBP was inhibited. It was also observed that pentose phosphates accumulated and the and NADPH/NADP⁺ ratio increased (FIG. 3D), indicating a relatively higher pentose phosphate pathway flux. These metabolome data suggested that either phosphoglucose isomerase (Pgi1 in yeast) or phosphofrutokinase (Pfk1,2 in yeast) was inhibited.

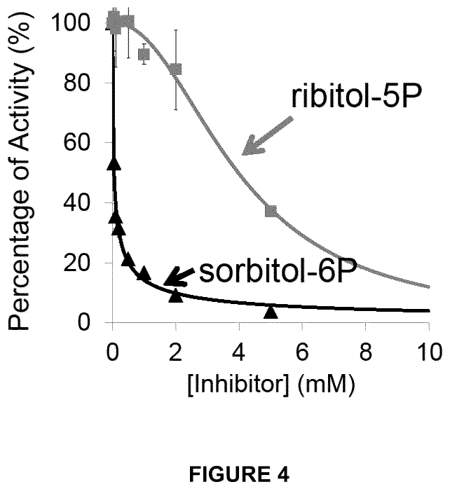

The mechanism of phosphofructokinase involves the phosphorylation of the β-furanose form of F6P into FBP without opening of the furanose ring. The mechanism of phosphoglucose isomerase involves the opening of the glucose pyranose ring, isomerization of glucose into fructose through an enediol intermediate, and the closing of the fructose ring. It was determined that the structure of sorbitol-6P mimics the structure of the enediol intermediate of phosphoglucose isomerase (FIG. 4A), indicating that sorbitol-6P is likely to be an inhibitor of this enzyme. Indeed, sorbitol-6P and other compounds with structures mimicking the enediol intermediate were previously reported as strong inhibitors of phosphoglucose isomerase in vitro. To confirm this, biochemical assays of purified Pgi1 were performed in the absence or presence of different concentrations of sorbitol-6P. It was determined that sorbitol-6P is a very strong inhibitor of Pgi1 activity with an IC₅₀ of ˜50 μM (FIG. 4B). Upon adding 1 mM sorbitol-6P, Pgi1 activity was inhibited >85%. Such inhibition resulted in the hyperactive oxidative PPP flux and hypoactive glycolytic flux observed in yA6PR cells and the growth defect in pyp1Δ cells grown on sorbitol. Because phosphoglucose isomerase was previously not considered as a key regulatory point of glycolysis, its capability of changing glycolytic rate is surprising. To further confirm that inhibition of Pgi1 would slow down glycolytic flux and upregulate oxidative PPP flux, Pgi1 was expressed under an inducible promoter. Indeed, lower induction of Pgi1 resulted in a similar metabolic phenotype in glycolysis as the yA6PR strain, confirming the physiological significance of phosphoglucose isomerase in maintaining glycolytic rate (FIG. 3F).

### Example 5

### Pyp1 Maintains Pgi1 Activity According to Cell's Growth Rate

To test if the inhibition of glycolysis is conserved among other polyol species and if Pyp1 is also responsive for protecting glycolysis from other polyol phosphates, wild type and pyp1Δ cells were grown on glycerol or mannitol. Indeed, pyp1Δ cells grew defectively on glycerol and were unable to grow on mannitol, further confirming that Pyp1 is a D-polyol phosphatase in vivo.

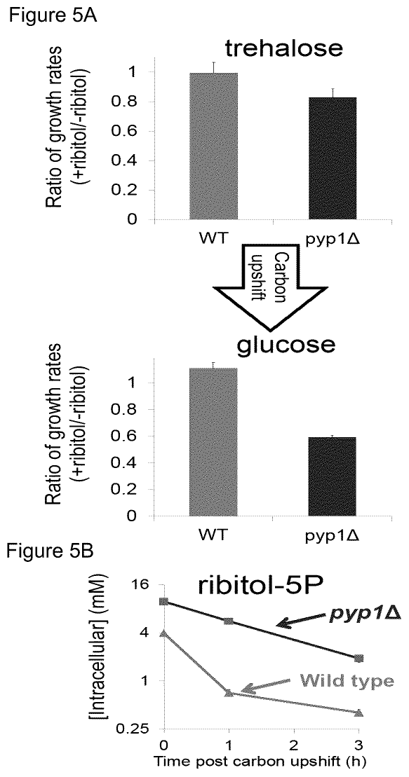

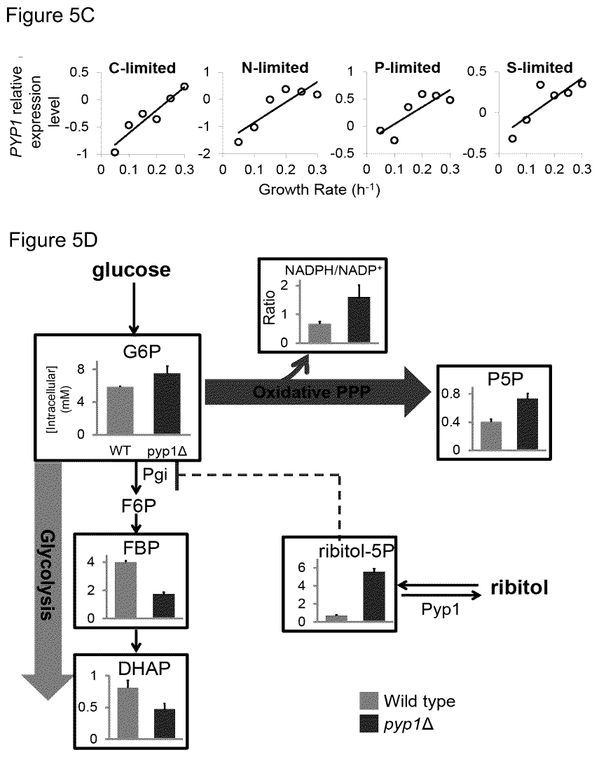

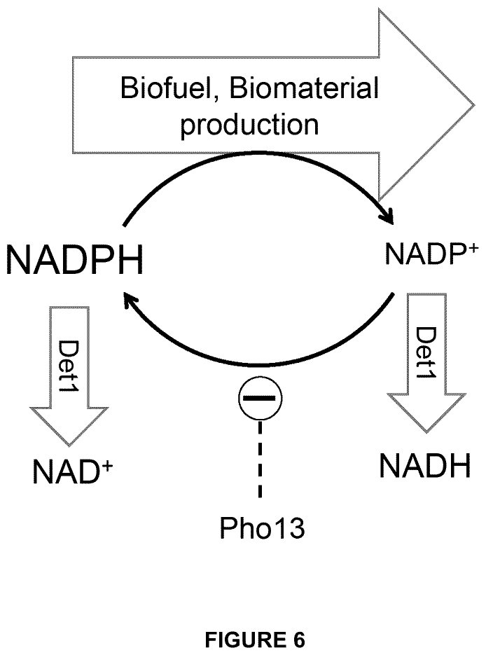

Other polyols, such as erythritol, ribitol and xylitol also exist widely in nature. Because yeast cannot utilize these polyols as a sole carbon source, a condition where these polyols could be transported and phosphorylated at a high rate in growing cells was sought. Trehalose is a natural dimer of glucose that can be slowly utilized by yeast by its cleavage into two glucose monomers by the enzyme trehalase. The cleavage and growth rate is slow, so the growth is usually considered a glucose-limited culturing condition. It was determined that in the presence of ribitol, ribitol-5P accumulates, but does not affect the growth rate of wild type cells growing on trehalose (FIGS. 5A, upper panel; and B, time zero). In contrast, the presence of ribitol resulted in a ˜2.5-fold higher accumulation of ribitol-5P and a ˜20% decrease of the growth rate of pyp1Δ cells (FIGS. 5A, upper panel; and B, time zero). Biochemical assays of Pgi1 against different concentrations of ribitol-5P were also performed (FIG. 3B). Although ribitol-5P did not inhibit Pgi1 as strongly as sorbitol-6P, its much higher intracellular level still resulted in a strong inhibitory effect.

Since all of the above results indicate that Pyp1 is essential for yeast in the presence of polyols in relatively poor growth conditions, Pyp1 expression levels were determined at different growth rates controlled by chemostats. Surprisingly, Pyp1's expression is highly correlated to growth rate, regardless of the limiting nutrient used (FIG. 5C). To address this paradox, the mechanism of polyol phosphate inhibition on cell growth was investigated. Phosphoglucose isomerase is a reversible enzyme that usually has high expression level compared to irreversible enzymes (Bennett et al., Nature Chemical Biology 2009, 5, 593). The reaction net flux could also be easily modulated by the concentration of its substrate and product. Such features render phosphoglucose isomerase activity difficult to completely inhibit, which was also the reason why it was not considered as the key regulatory point. As a result, slower growing cells with smaller demand for glycolytic flux might not be affected as much as faster growing cells in which high glycolytic flux needs to be maintained.

To test this hypothesis, pyp1Δ cells and wild type cells were grown on trehalose in the presence of ribitol and then switched to glucose plus ribitol. This carbon upshift boosted the cell's growth rate. Although the transport of ribitol into the cell is repressed by glucose, the accumulated ribitol-5P could not be degraded instantly in pyp1Δ cells, resulting in an eight-fold higher concentration of ribitol-5P compared to wild type cells after one hour of switching to glucose (FIG. 5B, time=1h). The accumulated ribitol-5P resulted in ˜50% lower growth rate in pyp1Δ cells, which is much greater than the growth defect seen in slower growth conditions. In comparison, the growth of wild type cells in the same conditions was not affected (FIG. 5A, lower panel). Metabolome profiling indicates a strong inhibition of Pgi1 upon carbon upshift (FIG. 5D). These results together proved that Pyp1 was indeed more important at higher growth rates due to the higher demand for driving glycolysis.

### Example 6

### Deletion of DET1 and PHO13 Results in Accumulation of NADPH. Deletion of DET1 Results in Accumulation of NADP+.

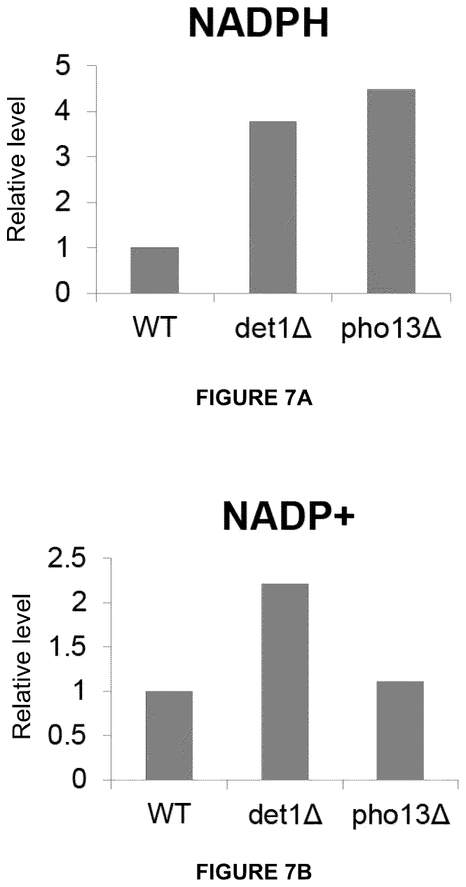

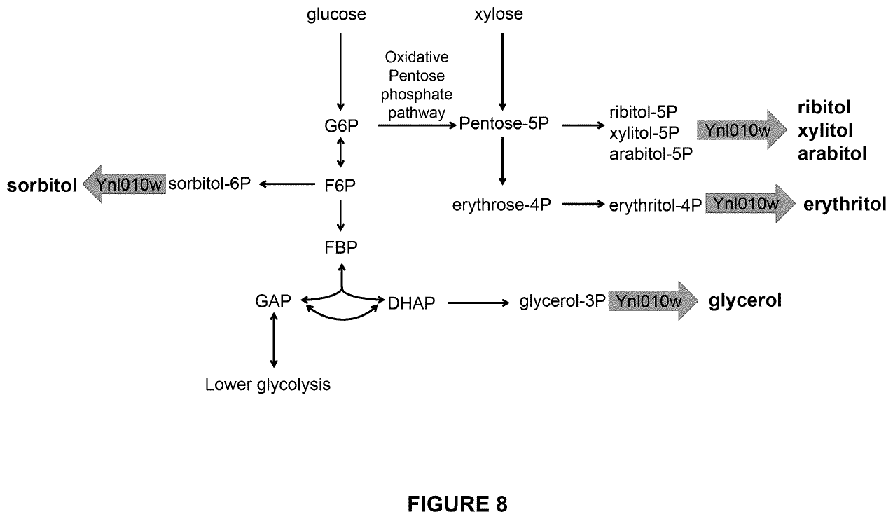

NADPH and NADP+ levels in S. cerevisiae cells lacking either DET1 or PHO13 were compared to NADPH and NADP+ levels in wild type cells. The results showed that deletion of DET1 or PHO13 accumulates NADPH in S. cerevisiae cells. Deletion of DET1, but not PHO13, also accumulates NADP+ in S. cerevisiae cells (FIG. 7).

### Materials and Methods

Cellular NADPH and NADP+ levels were quantitated by liquid chromatography-mass spectrometry. det1 and pho13 deletion strains were created by homologous recombination using the allele amplified by PCR from the synthetic genetic analysis (SGA) deletion set.⁴⁷ Cells were grown in minimal media comprising 6.7 g/L Difco Yeast Nitrogen Base without amino acids plus 2% (w/v) glucose. The metabolome of batch culture Saccharomyces cerevisiae was characterized as described previously. Briefly, saturated overnight cultures were diluted 1:30 and grown in liquid media in a shaking flask to A600 of ˜0.6. A portion of the cells (3 mL) were filtered onto a 50 mm nylon membrane filter (Millipore, Billerica, MA), which was immediately transferred into −20° C. extraction solvent (40:40:20 acetonitrile/methanol/water).

LC/MS Metabolite measurement Cell extracts were analyzed by reversed phase ion-pairing liquid chromatography (LC) coupled by electrospray ionization (ESI) (negative mode) to a high-resolution, high-accuracy mass spectrometer (Exactive; Thermo Fisher Scientific, Waltham, Massachusetts) operated in full scan mode at 1 s scan time, 10⁵ resolution. Relative concentration of NADPH and NADP⁺ between wild-type, det1 and pho13 strains were determined using the in-house developed, open-source software MAVEN. Compounds' identities were verified by mass and retention time matched to authenticated standards. Isomers are reported separately only where they are fully chromatographically resolved.

### Example 7

Additional experiments were run to confirm the results above, as well as investigate new characteristics of the gene.

Methods: Protein Purification and Enzymatic Assays

For Pyp1's polyol phosphatase activity assay, a yeast Open Reading Frame (ORF) strain with an expression vector containing C-terminal His-tagged PYP1 (Open Biosystems, Thermo Fisher Scientific, San Jose, CA) was grown on galactose to induce Pyp1 expression. The resulting cells were lysed using glass beads and His-tagged Pyp1 was purified using Qiagen Ni-NTA spin columns according to the manufacturer's instructions. Phosphatase activity against ribitol-5-phosphate, sorbitol-6-phosphate and octulose-8-phosphate was determined by monitoring the increase of ribitol, sorbitol or octulose using LC-MS. The reaction mixture contained 100 mM Tris-HCl at the pH 8.0, 10 mM MgCl₂ and a range of substrate concentrations (0.01 to 5 mM).

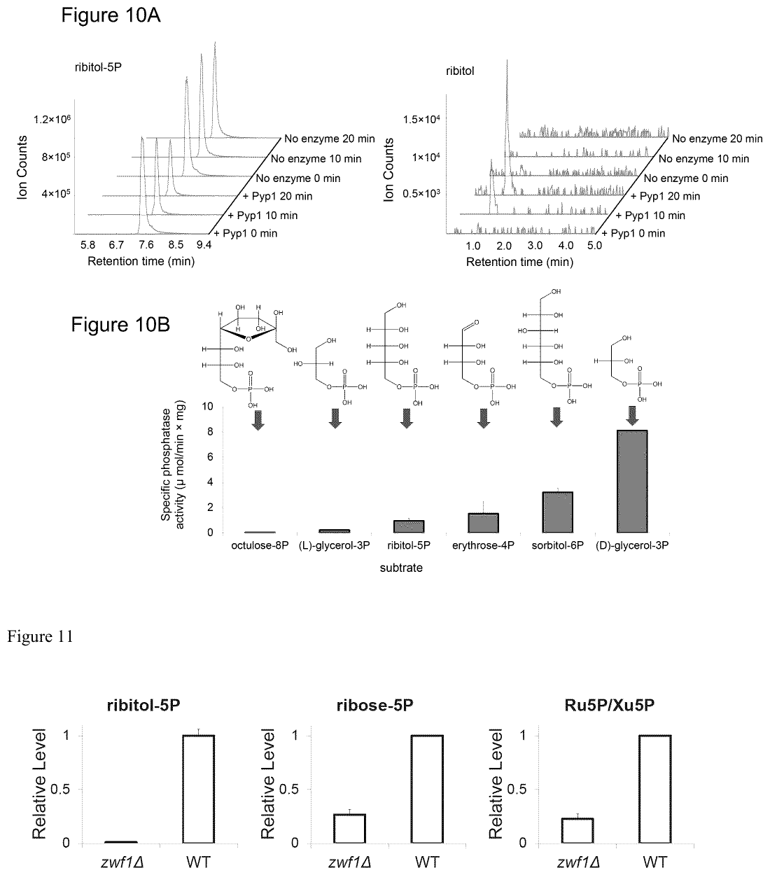

Purified Pyp1 was also assayed against other compounds with similar structures and 90 common phosphorylated metabolites. Briefly, the gene encoding Pyp1 was amplified by PCR using S. cerevisiae genomic DNA. The amplified fragments were cloned into a modified pET15b vector (Novagen, Darmstadt, Germany) and overexpressed in the E. coli BL21 (DE3) Gold strain (Stratagene, La Jolla, California) as previously described (Kuznetsova et al., 2010). The recombinant protein was purified using metal ion affinity chromatography on nickel chelate resin (Qiagen, Hilden, Germany) to high homogeneity and stored at −80° C. Purified Pyp1 was then screened for phosphatase activity as described previously (Kuznetsova et al., 2006). Due to the difficulty of obtaining (D)-glycerol-3P, the activity of Pyp1 on (D)-glycerol-3P is determined using racemic glycerol-3P as the substrate. Because (L)-glycerol-3P has very low activity, the resulting activity on racemic glycerol-3P was used as the activity on (D)-glycerol-3P. Compounds with specific activity higher than 0.1 mol/mg/min are shown in FIG. 10B.

Results

To measure the inhibition of Pgi by sorbitol-6-phosphate and ribitol-5-phosphate, yeast Pgi1 was purchased from Sigma Aldrich (St. Louis, MO). Phosphoglucose isomerase activity was determined by adding fructose-6-phosphate and monitoring the appearance of glucose-6-phosphate using LC/MS. We found such LC/MS based assay is consistently more sensitive and accurate than the more typical colorimetric-based assay, which involves coupling the Pgi1 activity with glucose-6-phosphate dehydrogenase activity and monitoring the appearance of NADH. The reaction mixture contained 100 mM Tris-HCl at pH 8.0, 10 mM MgCl₂, 0.6 mM fructose-6-phosphate (the physiological concentration in cells grown exponentially on glucose), and a range of sorbitol-6-phosphate and ribitol-5-phosphate concentrations (0.05 to 5 mM). The resulting data were fitted to the Hill equation using the GraphPad Prism Software.

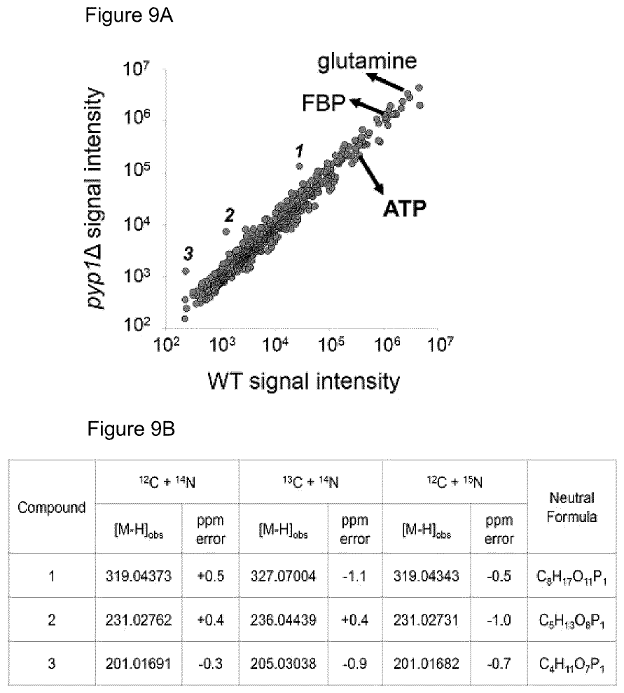

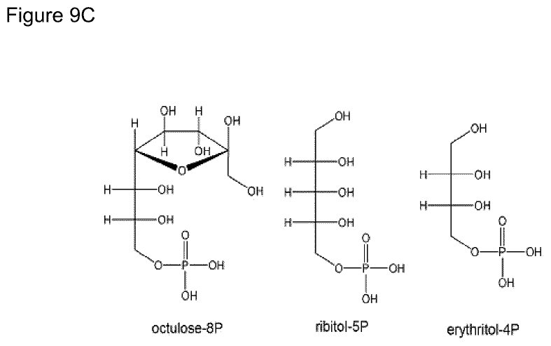

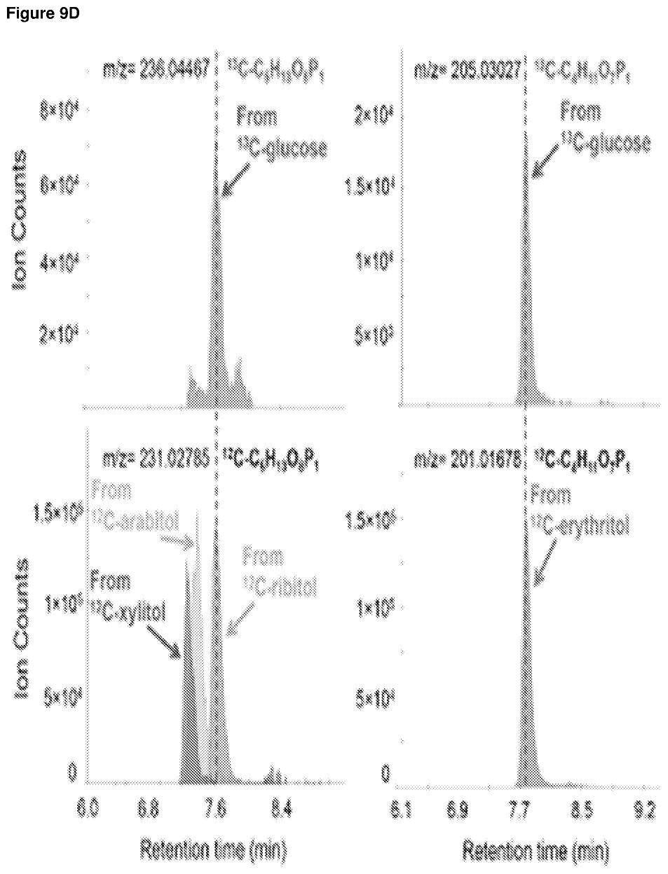

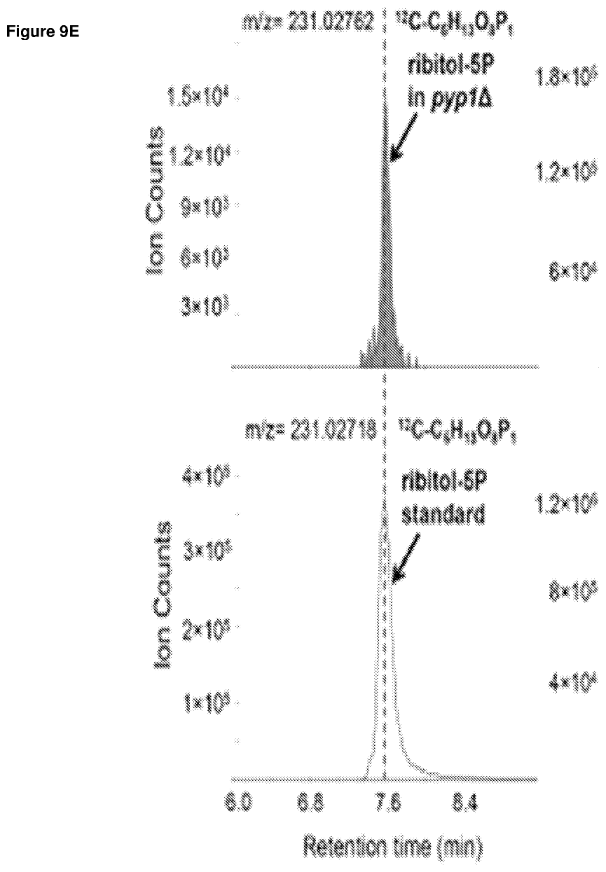

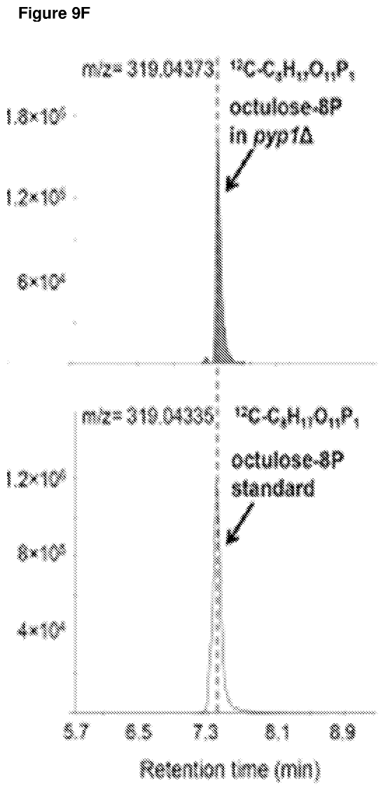

Yeast strains with putative phosphatases of unknown function deleted for changes in metabolite concentrations were screened. Yeast were grown in glucose minimal media, and metabolites were extracted into 40:40:20 methanol: acetonitrile: water, followed by metabolome analysis by reversed-phase ion-pairing liquid chromatography-high resolution mass spectrometry (LC-MS). It was found that the deletion of PYP1, formerly known as the uncharacterized gene YNL010W, while not significantly altering the concentrations of most metabolites, led to the statistically significant (false discovery rate <0.05) accumulation of three compounds in negative ion mode (FIG. 9A). The metabolites' formulae were obtained by labeling cells with ¹³C and ¹⁵N and observing no shift from nitrogen labeling and a shift of +8, +5, or +4 daltons from carbon labeling (FIG. 9B) (Hegeman et al., 2007). Exact masses of these compounds matched putative formulae of C₈H₁₇O₁₁P₁, C₅H₁₃O₈P₁ and C₄H₁₁O₇P₁. Searching for these formulae in the KEGG database returned the metabolites octulose-8-phosphate (C₈H₁₇O₁₁P₁); ribitol-5-phosphate, arabitol-5-phosphate, and xylitol-5-phosphate (C₅H₁₃O₈P₁); and erythritol-4-phosphate (C₄H₁₁O₇P₁) (FIG. 9C).

To identify the five- and four-carbon polyol phosphates, we performed an isotope labeling experiment involving switching pyp1Δ cells from U—¹³C-glucose to unlabeled ribitol, arabitol, xylitol or erythritol. Although baker's yeast cannot effectively utilize these polyols as carbon sources, in the absence of glucose, they slowly transport and phosphorylate them. Results showed that feeding ribitol and erythritol resulted in the build-up of intracellular metabolites with exact mass and LC retention time matching the C₅H₁₃O₈P₁ and C₄H₁₁O₇P₁ that accumulated with Pyp1 deletion (FIG. 9D). Feeding of arabitol and xylitol resulted in the accumulation of polyol phosphates with different retention times (FIG. 9D). Based on these results, ribitol-5P was synthesized and confirmed that exact mass and retention time matched to the endogenous 5-carbon sugar alcohol (FIG. 9E). Octulose-8P has been reported in yeast previously (Clasquin et al., 2011), and the chromatographic retention time of the accumulated C₈H₁₇O₁₁P₁ exactly matched the synthetic octulose-8P standard (FIG. 9F). Thus, the compounds that accumulate in the pyp1Δ strain are octulose-8P, ribitol-5P and erythritol-4P.

Ribitol-5P and erythritol-4P are polyol phosphates, but octulose-8P is a sugar phosphate. We were curious why Pyp1 deletion resulted in accumulation of metabolites from these different structural families. The most common conformer of the seven carbon sugar sedoheptulose and its derivatives in solution is β-furanose (Kuchel et al., 1990). Octulose-8P likely has a similar β-furanose ring structure (FIG. 9C), which has three carbons (6′-8′) of octulose out of the ring, forming a tail that resembles (D)-glycerol-3P. Note that (D)-glycerol-3P is more commonly referred to as (L)-glycerol-1P. We use the (D)-glycerol-3P nomenclature to emphasize the structural similarity to longer (D)-polyol phosphates, including ribitol-5P and erythritol-4P (FIG. 9C). On this basis, it was hypothesized that Pyp1 is a broad spectrum polyol phosphatase that non-specifically hydrolyzes phosphate from compounds containing a (D)-glycerol-3P substructure.

Pyp1 is a Broad Spectrum (D)-polyol Phosphatase

Although polyols are excretion products of fungi and plants, polyol phosphates were not previously thought to be an intermediate in this pathway (sugar phosphate→sugar→polyol). To determine if the accumulated compounds were indeed the substrates of Pyp1, the biochemical activity of the purified recombinant Pyp1 on these compounds was analyzed. Incubation with Pyp1 led to the depletion of ribitol-5P and the accumulation of ribitol (FIG. 10A), whereas no detectable phosphatase activity was found against octulose-8P (FIG. 10B). Because flux through octulose-8P is very low in cells, minimal Pyp1 activity may nevertheless be sufficient to alter the cellular concentration (Clasquin et al., 2011). Alternatively, Pyp1 may need to bind an activator or other substrate in order to hydrolyze octulose-8P. To better define the range of substrates of Pyp1, its activity was also measured against erythrose-4P, sorbitol-6P, racemic glycerol-3P, and (L)-glycerol-3P. Incubation with Pyp1 led to the hydrolysis of erythrose-4P, sorbitol-6P and racemic glycerol-3P, but not (L)-glycerol-3P (FIG. 10B). Thus, unlike the known glycerol-3-phosphatases (Hor2 and Rhr2) in the glycerol biosynthetic pathway, which act on both (L) and (D)-glycerol-3P (Norbeck et al., 1996), Pyp1 is specific to (D)-polyol phosphates. No other phosphatase activity was found for other common phosphorylated compounds. Interestingly, the best biochemical substrates for Pyp1 do not match those that accumulated in cells, likely because cellular accumulation depends on the absence of other routes of metabolizing the compounds.

Source of Ribitol-5P in Glucose-Grown Yeast

Although polyol phosphates have not been previously described in Baker's yeast, they have been noted in other fungi species (Jennings, 1984). There are two likely routes for the cellular biosynthesis of these compounds. The first involves the reduction of the corresponding sugar phosphate and the second involves phosphorylation of polyols. Both activities have been described in fungi but the responsible genes remain unknown (Jennings, 1984). In Baker's yeast grown on glucose, the only known polyol is glycerol, and thus polyol phosphates are likely made from the reduction of sugar phosphates (as in Haemophilus influenza, Staphylococcus aureus (Pereira and Brown, 2004), and Escherichia coli (Novotny et al., 1984)). Consistent with this, deletion of Zwf1, the first step in the oxidative pentose phosphate pathway (PPP), eliminated the endogenous peak for ribitol-5P, presumably due to decreased levels of ribose-5P and ribulose-5P (FIG. 11). This is consistent with ribitol-5P being made via the reduction of ribose-5P or ribulose-5P in yeast grown on glucose.

Growth on Sorbitol Requires Pyp1

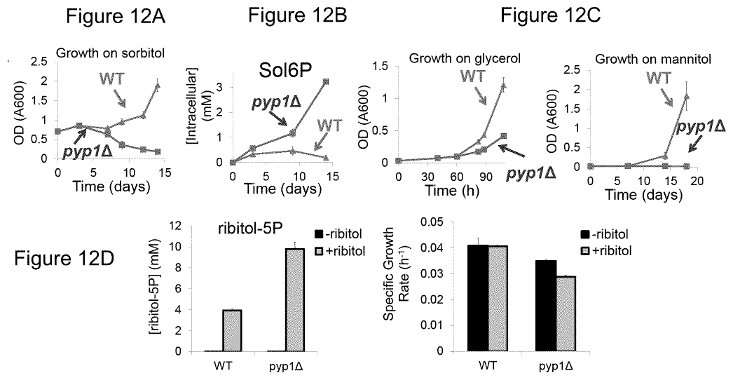

When polyols are present in the growth environment, polyol phosphates can be made by their phosphorylation. Of the natural long-chain polyols, sorbitol is the only one known to support yeast growth as the sole carbon source (Sarthy et al., 1994). The very long lag of growth begins with sorbitol dehydrogenase which converts sorbitol to fructose. Due to the structural similarity of sorbitol to glucose, we hypothesize that yeast might sometimes erroneously phosphorylate sorbitol into sorbitol-6P, which could be toxic in excess. To test this, both wild type and pyp1Δ strains were fed minimal media containing sorbitol as the sole carbon source. While wild type cells grew to saturation after a long lag phase, pyp1Δ cells never grew (FIG. 12A). Metabolome profiling revealed that sorbitol-6P levels in the pyp1Δ strain were much higher than the wild type strain (FIG. 12B). These results are consistent with Pyp1 being required to dephosphorylate sorbitol-6P, which otherwise accumulates to toxic levels.

To investigate whether Pyp1 is important for growth on other polyol substrates, pyp1Δ yeast were fed glycerol or mannitol. The pyp1Δ cells grew poorly on glycerol. While wild type cells managed to reach saturation after a long lag phase on mannitol, pyp1Δ cells were unable to grow on mannitol (FIG. 12C). Thus, Pyp1 broadly contributes to polyol phosphate detoxification, and such detoxification is required for growth on diverse polyol substrates.

To more directly ascertain whether the impaired growth was a result of polyol phosphate toxicity, yeast trehalose (a dimer of glucose that can be slowly utilized by yeast resulting in carbon-limited growth) (Jules et al., 2004; Walther et al., 2010)) were fed with or without addition of ribitol. In wild type yeast, addition of ribitol to the medium resulted in accumulation of intracellular ribitol-5P, without impacting growth rate (FIG. 12D). In pyp1 yeast, the ribitol-5P levels rose yet higher, resulting in a ˜20% decrease in the growth rate (FIG. 12D). Thus, buildup of polyol phosphate compounds impairs yeast growth, including on substrates other than polyols.

Polyol Phosphates are Inhibitors of Phosphoglucoisomerase (Pgi)

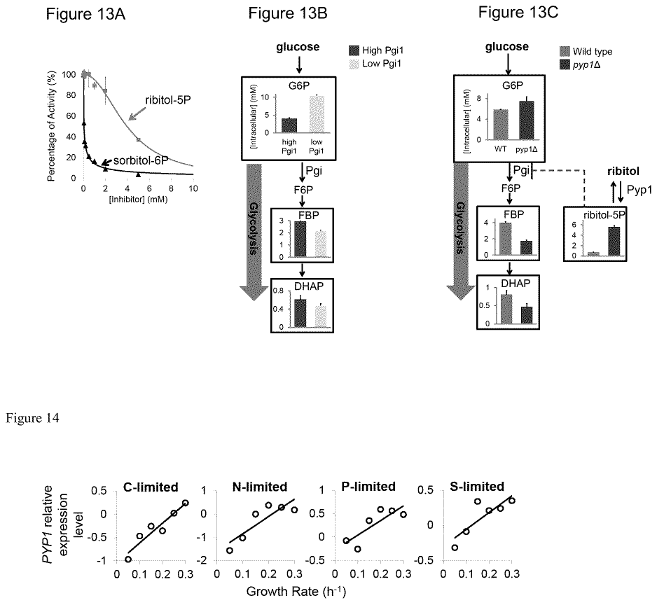

One potential mechanism by which polyol phosphates could impair cell growth is through metabolic enzyme inhibition. Sorbitol-6P is structurally similar to the enediol transition state of the Pgi reaction (Scheme 1) and sorbitol-6P and other structural mimics of the enediol intermediate are known Pgi inhibitors (Milewski et al., 2006). It was confirmed that sorbitol-6P and less potently ribitol-5P inhibit Pgi (FIG. 13A). Motivated by these observations, it was sought to determine whether polyol phosphates significantly and selectively inhibit Pgi in yeast cells. It was hypothesized that two metabolic hallmarks of Pgi inhibition would be increased glucose-6P and decreased glycolytic intermediates downstream of fructose-6P, with fructose-1, 6-bisphosphate (FBP) and dihydroxyacetone phosphate (DHAP) convenient marker compounds. To evaluate whether decreased Pgi activity indeed induces these metabolic changes, constructed a yeast strain with PGI under control of an estradiol-inducible promoter was constructed. In the low induction condition (1 nM estradiol, protein level ˜1/7 of WT cells or 100 nM induction), both hallmarks of low Pgi activity were observed (FIG. 13B).

It was then tested whether ribitol-5P accumulation results in these metabolic hallmarks of Pgi deficiency. Cells were initially grown in trehalose+ribitol and then switched to glucose+ribitol to enhance glycolytic flux. Relative to wild-type yeast, the pyp1Δ strain accumulated dramatically more ribitol-5P (FIG. 13C). Critically, the yeast lacking Pyp1 also manifested both hallmarks of physiological Pgi inhibition (FIG. 13C).

Discussion

Sorbitol-6-phosphatase activity has been found in apple leaves (Zhou et al., 2003) and silk worms (Oda et al., 2005). The gene encoding this enzyme, however, has not been discovered. In engineered fungi, sorbitol production has been achieved by expressing bacterial sorbitol-6P dehydrogenase, but again the gene encoding the required phosphatase activity remained missing (Ladero et al., 2007). Here we identify the previously unannotated yeast gene YNL010W, which we now term PYP1, as a polyol phosphate phosphatase. Pyp1 dephosphorylates a variety of compounds with the common structural motif of a D-glycerol-3-phosphate tail, including D-glycerol-3P, erythrose-4P, ribitol-5P, and sorbitol-6P (FIG. 2B). It is likely that Pyp1 would also dephosphorylate erythritol-4P, arabitol-5P, and xylitol-5P. The ability of Pyp1 to hydrolyze a variety of sugar alcohol phosphates gives rise to an intriguing opportunity to employ Pyp1 in the production of diverse sugar alcohol consumables.

One functional role of Pyp1 is to limit the polyol phosphate concentrations in cells. High levels of polyol phosphates impair yeast growth, at least in part by inhibition of the upper glycolytic enzyme phosphoglucoisomerase (Pgi), for which sorbitol-6-phosphate is a transition state analogue. Other sugar phosphate isomerases, such as triose-phosphate isomerase and ribose-5-phosphate isomerase, have similar enediol reaction intermediates (Komives et al., 1991; Zhang et al., 2003), and thus are also expected to be inhibited by polyol phosphates. Similar to strong inhibition of Pgi by sorbitol-6 phosphate, triose-phosphate isomerase will likely be strongly inhibited by D-glycerol-3P, and ribose-5-phosphate isomerase by ribitol-5P or xylitol-5P. Flux through each of these enzymes tends to increase with faster yeast growth rate. For example, ribose-5-phosphate isomerase is required to feed ribosome biogenesis. Thus rapidly growing yeast cells may be particularly sensitive to enzyme inhibition by polyol phosphates. To determine whether Pyp1 function is associated with growth rate, we analyzed its expression as a function of growth rate across 25 different chemostat conditions (Brauer et al., 2008). Interestingly, Pyp1's expression was strongly positively correlated with growth rate, regardless of the limiting nutrient used (top 6% of all transcripts in genome) (FIG. 14). Thus, unlike most protective or detoxification genes (e.g. against heat, osmolarity or oxidative stress), which are highly expressed under slower growth conditions (Brauer et al., 2008; Gasch et al., Molecular biology of the cell 11, 4241-4257, 2000), PYP1 is a fast-growth gene that maintains high Pgi flux by dephosphorylating polyol phosphates.

The transition state inhibition by polyol phosphates and their derivatives is not limited to sugar phosphate isomerase. In plants, ribulose-1,5-bisphosphate carboxylase/oxygenase (RuBisCO) also has an enediol intermediate and is known to be inhibited by 2-carboxy-D-arabitol-1-phosphate (CA1P) (Andralojc et al., 1994). The corresponding 2-carboxy-D-arabitol-1-phosphatase is activated by light and controls cellular level of CA1P. In darkness, RuBisCO is inhibited and also protected by CA1P from proteolysis (Khan et al., FEBS 266, 840-847, 1999).

Pyp1 is conserved across fungi species and some plants and bacteria, including Ricinus communis, Dehalococcoides ethenogenes, and Bacillus anthracis (Gibney et al., Phylogenetic Portrait of the Saccharomyces cerevisiae Functional Genome. G3 (Bethesda) 3, 1335-1340, 2013). The highly conserved nature of Pyp1 in fungi indicates the importance of clearing polyol phosphates. It is likely that the homologous enzymes play the same role in bacteria and plants. Although we have not identified a Pyp1 homolog in mammals, nor are linear polyol phosphates longer than three carbons known mammalian metabolites, polyols are also produced in humans and can accumulate in disease (Lee et al., 1995). For example, during hyperglycemia (e.g., due to diabetes), sorbitol accumulates in a number of organs due to the action of aldose reductase on glucose, which may contribute to diabetic complications including diabetic retinopathy, peripheral neuropathy and diabetic kidney disease (Brownlee, 2001; Nishikawa et al., 2000; Schrijvers et al., Endocrine reviews 25, 971-1010, 2004). It is unclear whether such sorbitol sometimes becomes phosphorylated to sorbitol-6P and, if so, whether sorbitol-6P contributes to disease pathology.

Beyond their potential cellular toxicity, polyol phosphates may also have a productive regulatory role in central carbon metabolism. Pgi sits at the branch point between glycolysis and the oxidative pentose phosphate pathway. Despite being ideally situated to regulate the branching ratio between these pathways, Pgi is not an allosteric enzyme, with no physiological regulators known (Milewski et al., 2006; Noltmann, 1972). It is tempting to speculate that polyol phosphates might serve as endogenous Pgi regulators, rendering Pgi an enzyme controlled by active site competition (Fell, 1997, Understanding the Control of Metabolism (Portland Press).; Goyal et al., PLoS computational biology 6, e1000802, 2010; Heinrich and Rapoport, European Journal of Biochemistry 42, 89-95, 1974; Hofmeyr and Cornishbowden, European Journal of Biochemistry 200, 223-236, 1991; Kacser et al., Biochemical Society Transactions 23, 341-366, 1995; Kell and Westerhoff, Fems Microbiology Reviews 39, 305-320, 1986). It is hypothesized that polyol phosphates, and their hydrolysis by Pyp1 contribute to physiological metabolic flux control.

### REFERENCES

(1) Teo, G.; Suzuki, Y.; Uratsu, S. L.; Lampinen, B.; Ormonde, N.; Hu, W. K.; DeJong, T. M.; Dandekar, A. M. Proc Natl Acad Sci USA 2006, 103, 18842.
(2) Shindou, T.; Sasaki, Y.; Miki, H.; Eguchi, T.; Hagiwara, K.; Ichikawa, T. J Food Hyg Soc Jpn 1988, 29, 419.
(3) Pahlman, A. K.; Granath, K.; Ansell, R.; Hohmann, S.; Adler, L. J Biol Chem 2001, 276, 3555.
(4) Nevoigt, E.; Stahl, U. FEMS Microbiol Rev 1997, 21, 231.
(5) Bradshaw, D. J.; Marsh, P. D. Caries Res 1994, 28, 251.
(6) Chang, Q.; Griest, T. A.; Harter, T. M.; Petrash, J. M. Bba-Mol Cell Res 2007, 1773, 321.
(7) Moon, H. J.; Jeya, M.; Kim, I. W.; Lee, J. K. Applied Microbiology and Biotechnology 2010, 86, 1017.
(8) Povelainen, M.; Miasnikov, A. N. Biotechnology journal 2006, 1, 214.
(9) Povelainen, M.; Miasnikov, A. N. Journal of biotechnology 2007, 128, 24.
(10) Toivari, M. H.; Maaheimo, H.; Penttila, M.; Ruohonen, L. Appl Microbiol Biotechnol 2010, 85, 731.
(11) Albertyn, J.; Hohmann, S.; Thevelein, J. M.; Prior, B. A. Mol Cell Biol 1994, 14, 4135.
(12) Norbeck, J.; Pahlman, A. K.; Akhtar, N.; Blomberg, A.; Adler, L. Journal of Biological Chemistry 1996, 271, 13875.
(13) Moreno-Sanchez, R.; Saavedra, E.; Rodriguez-Enriquez, S.; Olin-Sandoval, V. J Biomed Biotechnol 2008.
(14) Galazzo, J. L.; Bailey, J. E. Enzyme Microb Tech 1990,12, 162.
(15) Torres, N. V.; Mateo, F.; Melendezhevia, E.; Kacser, H. Biochemical Journal 1986, 234, 169.
(16) Small, J. R.; Kacser, H. European Journal of Biochemistry 1993, 213, 613.
(17) Vander Heiden, M. G.; Lunt, S. Y.; Dayton, T. L.; Fiske, B. P.; Israelsen, W. J.; Mattaini, K. R.; Vokes, N. I.; Stephanopoulos, G.; Cantley, L. C.; Metallo, C. M.; Locasale, J. W. Cold Spring Harbor symposia on quantitative biology 2011, 76, 325.
(18) Berg, J. M.; Tymoczko, J. L.; Stryer, L. In Biochemistry; W.H. Freeman and Co.: New York, 2007.
(19) Read, J.; Pearce, J.; Li, X. C.; Muirhead, H.; Chirgwin, J.; Davies, C. Journal of Molecular Biology 2001, 309, 447.
(20) Hegeman, A. D.; Schulte, C. F.; Cui, Q.; Lewis, I. A.; Huttlin, E. L.; Eghbalnia, H.; Harms, A. C.; Ulrich, E. L.; Markley, J. L.; Sussman, M. R. Anal Chem 2007, 79, 6912.
(21) Clasquin, M. F.; Melamud, E.; Singer, A.; Gooding, J. R.; Xu, X.; Dong, A.; Cui, H.; Campagna, S. R.; Savchenko, A.; Yakunin, A. F.; Rabinowitz, J. D.; Caudy, A. A. Cell 2011, 145, 969.
(22) Kuchel, P. W.; Berthon, H. A.; Bubb, W. A.; Mcintyre, L. M.; Nygh, N. K.; Thorburn, D. R. Biomed Biochim Acta 1990, 49, S105.
(23) Jennings, D. H. Adv Microb Physiol 1984, 25, 149.
(24) Pereira, M. P.; Brown, E. D. Biochemistry 2004, 43, 11802.
(25) Novotny, M. J.; Reizer, J.; Esch, F.; Saier, M. H. Journal of Bacteriology 1984, 159, 986.
(26) Sarthy, A. V.; Schopp, C.; Idler, K. B. Gene 1994, 140, 121.
(27) Postma, P. W.; Lengeler, J. W.; Jacobson, G. R. Microbiological Reviews 1993, 57, 543.
(28) Figueroa, C. M.; Iglesias, A. A. Biochimie 2010, 92, 81.
(29) Banaszak, K.; Mechin, I.; Obmolova, G.; Oldham, M.; Chang, S. H.; Ruiz, T.; Radermacher, M.; Kopperschlager, G.; Rypniewski, W. Journal of Molecular Biology 2011, 407, 284.
(30) Milewski, S.; Janiak, A.; Wojciechowski, M. Archives of Biochemistry and Biophysics 2006, 450, 39.
(31) Jules, M.; Guillou, V.; Francois, J.; Parrou, J. L. Appl Environ Microbiol 2004, 70, 2771.
(32) Walther, T.; Novo, M.; Rossger, K.; Letisse, F.; Loret, M. O.; Portals, J. C.; Francois, J. M. Mol Syst Biol 2010, 6, 344.
(33) Brauer, M. J.; Huttenhower, C.; Airoldi, E. M.; Rosenstein, R.; Matese, J. C.; Gresham, D.; Boer, V. M.; Troyanskaya, O. G.; Botstein, D. Molecular biology of the cell 2008, 19, 352.
(34) Bennett, B. D.; Kimball, E. H.; Gao, M.; Osterhout, R.; Van Dien, S. J.; Rabinowitz, J. D. Nature Chemical Biology 2009, 5, 593.
(35) Zhou, R.; Cheng, L. L.; Wayne, R. Plant Sci 2003, 165, 227.
(36) Oda, Y.; Iwami, M.; Sakurai, S. Insect Biochem Molec 2005, 35, 1284.
(37) Ladero, V.; Ramos, A.; Wiersma, A.; Goffin, P.; Schanck, A.; Kleerebezem, M.; Hugenholtz, J.; Smid, E. J.; Hols, P. APPLIED AND ENVIRONMENTAL MICROBIOLOGY 2007, 73, 1864.
(38) Lee, A. Y. W.; Chung, S. K.; Chung, S. S. M. Proceedings of the National Academy of Sciences of the United States of America 1995, 92, 2780.
(39) Nishikawa, T.; Edelstein, D.; Du, X. L.; Yamagishi, S.; Matsumura, T.; Kaneda, Y.; Yorek, M. A.; Beebe, D.; Oates, P. J.; Hammes, H. P.; Giardino, I.; Brownlee, M. Nature 2000, 404, 787.
(40) Brownlee, M. Nature 2001, 414, 813.
(41) Zhang, R.; Andersson, C. E.; Savchenko, A.; Skarina, T.; Evdokimova, E.; Beasley, S.; Arrowsmith, C. H.; Edwards, A. M.; Joachimiak, A.; Mowbray, S. L. Structure 2003, 11, 31.
(42) Komives, E. A.; Chang, L. C.; Lolis, E.; Tilton, R. F.; Petsko, G. A.; Knowles, J. R. Biochemistry 1991, 30, 3011.
(43) Kretschmer, M.; Fraenkel, D. G. Biochemistry 1991, 30, 10663.
(44) Noltmann, E. A. Aldose-ketose isomerases; Academic Press, 1972; Vol. 6.
(45) Pelicano, H.; Martin, D. S.; Xu, R. H.; Huang, P. Oncogene 2006, 25, 4633.
(46) Ralser, M.; Wamelink, M. M.; Struys, E. A.; Joppich, C.; Krobitsch, S.; Jakobs, C.; Lehrach, H. Proc Natl Acad Sci USA 2008, 105, 17807.
(47) Tong, A. H.; Evangelista, M.; Parsons, A. B.; Xu, H.; Bader, G. D.; Page, N.; Robinson, M.; Raghibizadeh, S.; Hogue, C. W.; Bussey, H.; Andrews, B.; Tyers, M.; Boone, C. Science 2001, 294, 2364.
(48) McIsaac, R. S.; Oakes, B. L.; Wang, X.; Dummit, K. A.; Botstein, D.; Noyes, M. B. Nucleic Acids Res 2013, 41, e57.
(49) Xu, Y. F.; Zhao, X.; Glass, D. S.; Absalan, F.; Perlman, D. H.; Broach, J. R.; Rabinowitz, J. D. Molecular cell 2012, 48, 52.
(50) Lu, W. Y.; Clasquin, M. F.; Melamud, E.; Amador-Noguez, D.; Caudy, A. A.; Rabinowitz, J. D. Analytical Chemistry 2010, 82, 3212.
(51) Melamud, E.; Vastag, L.; Rabinowitz, J. D. Analytical Chemistry 2010, 82, 9818.
(52) Bennett, B. D.; Yuan, J.; Kimball, E. H.; Rabinowitz, J. D. Nature Protocols 2008, 3, 1299.
(53) Egan, W.; Schneerson, R.; Werner, K. E.; Zon, G. Journal of the American Chemical Society 1982, 104, 2898.
(54) Kapuscinski, M.; Franke, F. P.; Flanigan, I.; Macleod, J. K.; Williams, J. F. Carbohydrate research 1985, 140, 69.
(55) Kuznetsova, E.; Xu, L.; Singer, A.; Brown, G.; Dong, A.; Flick, R.; Cui, H.; Cuff, M.; Joachimiak, A.; Savchenko, A.; Yakunin, A. F. J Biol Chem 2010, 285, 21049.
(56) Kuznetsova, E.; Proudfoot, M.; Gonzalez, C. F.; Brown, G.; Omelchenko, M. V.; Borozan, I.; Carmel, L.; Wolf, Y. I.; Mori, H.; Savchenko, A. V.; Arrowsmith, C. H.; Koonin, E. V.; Edwards, A. M.; Yakunin, A. F. J Biol Chem 2006, 281, 36149.
(57) Moon, H. J., Jeya, M., Kim, I. W. & Lee, J. K. Biotechnological production of erythritol and its applications. Applied Microbiology and Biotechnology 86, 1017-1025 (2010).
(58) Povelainen, M. & Miasnikov, A. N. Production of D-arabitol by a metabolic engineered strain of Bacillus subtilis. Biotechnol J 1, 214-9 (2006).
(59) Povelainen, M. & Miasnikov, A. N. Production of xylitol by metabolically engineered strains of Bacillus subtilis. J Biotechnol 128, 24-31 (2007).
(60) Toivari, M. H., Maaheimo, H., Penttila, M. & Ruohonen, L. Enhancing the flux of D-glucose to the pentose phosphate pathway in Saccharomyces cerevisiae for the production of D-ribose and ribitol. Appl Microbiol Biotechnol 85, 731-9 (2010).
(61) Albertyn, J., Hohmann, S., Thevelein, J. M. & Prior, B. A. GPD1, which encodes glycerol-3-phosphate dehydrogenase, is essential for growth under osmotic stress in Saccharomyces cerevisiae, and its expression is regulated by the high-osmolarity glycerol response pathway. Mol Cell Biol 14, 4135-44 (1994).
(62) Norbeck, J., Pahlman, A. K., Akhtar, N., Blomberg, A. & Adler, L. Purification and characterization of two isoenzymes of DL-glycerol-3-phosphatase from Saccharomyces cerevisiae—Identification of the corresponding GPP1 and GPP2 genes and evidence for osmotic regulation of Gpp2p expression by the osmosensing mitogen-activated protein kinase signal transduction pathway. Journal of Biological Chemistry 271, 13875-13881 (1996).

1. A recombinant S. cerevisiae cell comprising a polynucleotide encoding Pyp1, ortholog thereof, or a variant of Pyp1 at least 70% identical to the amino acid sequence of SEQ ID NO: 1, wherein the polynucleotide is operably linked to a heterologous promoter.

2. A recombinant microorganism comprising a polynucleotide encoding Pyp1, ortholog thereof, or a variant of Pyp1 at least 70% identical to the amino acid sequence of SEQ ID NO: 1.

3. The recombinant microorganism of claim 2, wherein the microorganism is a fungus or a bacteria.

4. The recombinant microorganism of claim 3, wherein the fungus is S. cerevisiae.

5. The recombinant microorganism of claim 2, wherein the microorganism further comprises a polynucleotide encoding an enzyme with sugar phosphate dehydrogenase activity operably linked to a heterologous promoter.

6. A method of producing a polyol comprising:
(a) culturing a recombinant microorganism comprising a polynucleotide encoding Pyp1, ortholog thereof, or a variant of Pyp1 at least 70% identical to the amino acid sequence of SEQ ID NO: 1 under conditions effective to express Pyp1, the ortholog thereof, or the variant of Pyp1; and optionally
(b) separating the polyol from the culture.

7. (canceled)

8. The method of claim 6 or 7, wherein the microorganism is a fungus or a bacteria.

9. The method of claim 8, wherein the fungus is S. cerevisiae.

10. The method of claim 6, wherein the polyol is a four, five or six-carbon polyol.

11. The method of claim 10, wherein the polyol is selected from the group consisting of erythritol, ribitol, arabitol, mannitol and sorbitol.

12. (canceled)

13. The method of claim 6, wherein the recombinant microorganism further comprises a polynucleotide encoding an enzyme with sugar phosphate dehydrogenase activity operably linked to a heterologous promoter.

14. A recombinant microorganism comprising a mutation or deletion of the DET1 gene or ortholog thereof and a mutation or deletion of the PHO13 gene or ortholog thereof.

15-17. (canceled)

18. The recombinant microorganism of claim 14, wherein the microorganism is a fungus or a bacteria.

19. (canceled)

20. A method of producing an electron rich compound comprising:
(a) culturing a Saccharomyces cerevisiae cell comprising a deletion or mutation of the DET1 gene and/or PHO13 gene, under conditions that promote over-expression of one or both of (i) a polynucleotide encoding an enzyme with sugar phosphate dehydrogenase activity and a gene encoding a polyol phosphatase or (ii) and engineered biosynthetic pathway for the electron rich compounds; and optionally
(b) separating the electron rich compound from the culture.

21. The method of claim 20 wherein the electron rich compound is selected from the group consisting of polyols, butanol, isobutanol, fatty acids, fatty acid esters, long-chain fatty alcohols, biodiesel and biogas.

22. The method of claim 21 wherein the polyol comprises 4 or more carbon atoms.

23-27. (canceled)


SEQUENCE LISTING (4 sequences)

SEQ ID NO: 1 - protein, 241 aa, Saccharomyces cerevisiae
MVKAVIFTDFDGTVTLEDSNDYLTDTLGFGKEKRLKVFEGVLDDTKSFRQGFMEMLESIHTPFPECIKILEKKIRLDPGFKDTFEWAQENDVPVIVVSSGMKPIIKVLLTRLVGQESIHKIDIVSNEVEIDAHDQWKIIYKDESPFGHDKSRSIDAYKKKFESTLKAGEQRPVYFYCGDGVSDLSAAKECDLLFAKRGKDLVTYCKKQNVPFHEFDTFKDILASMKQVLAGEKTVAELMEN

SEQ ID NO: 2 - DNA, 726 bp, Saccharomyces cerevisiae
  source 1..726: mol_type = other DNA
atggtcaaagctgttatttttaccgatttcgacggtaccgttactttggaagattctaacgattatctgaccgatactttaggtttcggtaaggaaaaaagattaaaggtcttcgaaggcgtgttagatgatactaagtcttttaggcaaggtttcatggaaatgctggaatccatccacacccctttccctgagtgtatcaagatcttagaaaagaaaattcgattggatcctggtttcaaggatacattcgaatgggctcaagaaaatgatgtccctgtcatcgttgtttccagcggaatgaaaccaattatcaaggttttattgaccagattagttggacaagagtctattcacaaaattgacattgtttccaatgaagtggaaatcgatgcacatgatcaatggaaaatcatctataaggatgaaagtcccttcggacatgacaaatccagaagtatcgatgcttataaaaagaaatttgaatctaccttgaaagcaggtgaacaaagacccgtttacttttactgtggtgacggtgtttctgatctaagtgctgcaaaagaatgtgatttgctatttgccaaaagaggtaaagatttggtcacttattgtaaaaaacaaaacgtcccattccatgaattcgataccttcaaagacatcttggctagcatgaaacaagttctggctggtgaaaagacagtcgctgaattgatggaaaattag

SEQ ID NO: 3 - DNA, 939 bp, Saccharomyces cerevisiae
  source 1..939: mol_type = other DNA
atgactgctcaacaaggtgtaccaataaagataaccaataaggagattgctcaagaattcttggacaaatatgacacgtttctgttcgattgtgatggtgtattatggttaggttctcaagcattaccatacaccctggaaattctaaaccttttgaagcaattgggcaaacaactgatcttcgttacgaataactctaccaagtcccgtttagcatacacgaaaaagtttgcttcgtttggtattgatgtcaaagaagaacagattttcacctctggttatgcgtcagctgtttatattcgtgactttctgaaattgcagcctggcaaagataaggtatgggtatttggagaaagcggtattggtgaagaattgaaactaatggggtacgaatctctaggaggtgccgattccagattggatacgccgttcgatgcagctaaatcaccatttttggtgaacggccttgataaggatgttagttgtgttattgctgggttagacacgaaggtaaattaccaccgtttggctgttacactgcagtatttgcagaaggattctgttcactttgttggtacaaatgttgattctactttcccgcaaaagggttatacatttcccggtgcaggctccatgattgaatcattggcattctcatctaataggaggccatcgtactgtggtaagccaaatcaaaatatgctaaacagcattatatcggcattcaacctggatagatcaaagtgctgtatggttggtgacagattaaacaccgatatgaaattcggtgttgaaggtgggttaggtggcacactactcgttttgagtggtattgaaaccgaagagagagccttgaagatttcgcacgattatccaagacctaaattttacattgataaacttggtgacatctacaccttaaccaataatgagttatag

SEQ ID NO: 4 - DNA, 1005 bp, Saccharomyces cerevisiae
  source 1..1005: mol_type = other DNA
atgtgtgaagagaatgttcatgttagtgaagatgttgctggcagtcacggttcttttacgaatgccagacctaggttaattgtactaataaggcatggggaaagcgaatcaaataaaaacaaggaggtcaatggttatattcctaaccatctgatttctttaacgaaaacaggtcaaatccaagctagacaagctggtattgacttattacgtgttttaaacgtagacgatcacaacttggtggaggatttggctaagaagtatattaaagatgaaagtagcaggagaactttaccgctgaaggactataccaggctgagtagagaaaaagacacaaacatagttttttatacatcaccctatagaagagcaagggaaacattgaaaggtattttggacgtcatcgatgaatataatgaattaaacagtggtgttcgtatatgtgaagatatgagatatgatccacatggtaaacagaaacatgcattttggccgagaggacttaataatactggtggtgtttacgaaaataatgaagataatatttgtgaagggaagcctggaaaatgttatctgcaatatcgggttaaggatgagccaagaataagggaacaagattttggtaatttccaaaaaatcaatagcatgcaggacgttatgaagaagagatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatag